(19) United States
(12) Patent Application Publication
(10) Pub. No.: US 2024/0034786 A1
(43) Pub. Date: Feb. 1, 2024

(54) AN ANTIBODY AGAINST P-CADHERIN AND USES THEREOF

(71) Applicant: WUXI BIOLOGICS IRELAND LIMITED, DUNDALK, CO LOUTH (IE)

(72) Inventors: Yuhong SHEN, Shanghai (CN); Jing LI, Shanghai (CN); Jie LI, Shanghai (CN)

(21) Appl. No.: 18/265,338

(22) PCT Filed: Dec. 9, 2021
(86) PCT No.: PCT/CN2021/136609

§ 371 Date: Jun. 5, 2023

(30) Foreign Application Priority Data
Dec. 10, 2020 (WO) PCT/CN2020/135185

(51) Int. Cl. C07K 16/28; C12N 15/63; A61P 35/00

(52) U.S. Cl. CPC C07K 16/28; C12N 15/63; A61P 35/00; C07K 2317/565; C07K 2317/567; C07K 2317/92; C07K 2317/24; C07K 2317/14

(57) ABSTRACT

Provided in the present disclosure are anti-P-cadherin antibodies, the nucleic acid molecules encoding the anti-P-cadherin antibodies, expression vectors and host cells used for the expression of anti-P-cadherin antibodies. The disclosure further provides the methods for validating the function of antibodies and uses thereof.

### CROSS REFERENCE

This application claims priority to International Patent Application No. PCT/CN2020/135185, filed on Dec. 10, 2020, the entire contents of which are incorporated herein by reference.

### SEQUENCE LISTING

The instant application contains a sequence listing which is hereby incorporated by reference in its entirety.

### FIELD

This application generally relates to antibodies. More specifically, the application relates to fully human monoclonal antibodies against P-cadherin, a method for preparing the same, and the use of the antibodies.

### BACKGROUND

Cadherins family proteins mediate cell-cell adhesions by homophilic interactions between two cadherin molecules at the surface of the respective cells in a cis and/or trans-manner and the cadherin-catenin complex constitutes the main building block of the adherens-type junctions. These complexes also represent a major regulatory mechanism that guides cell fate decisions, influencing cell growth, differentiation, cell motility and survival (Cavallaro and Dejana, Adhesion molecule signalling: not always a sticky business. Nat Rev Mol Cell Biol. 2011 March; 12 (3):189-97).

Cadherins may signal indirectly by recruiting signalling proteins, including β-catenin, p120 catenin (p120) and junction plakoglobin (plakoglobin) to the membrane; besides, Cadherins may form signalling units by interacting with growth factor receptors (eg. VEGFR2, EGFR, FGFR, PDGFR and TGFβ), forming signalling units with intracellular signalling partners such as kinases (eg. SRC family kinases, CSk) or phosphatases (eg. DEP1, SHP2, VE-PTP), or interacting with adaptor proteins (eg. SHC family members) through cadherins. After MMP (matrix metalloprotease) or ADAM (a member of the disintegrin and metalloprotease) family protease-mediated shedding of the ectodomain, intracellular proteases such as caspases and γ-secretase cleave the cadherin cytoplasmic tail, which may then translocate to the nucleus and regulate transcription (Cavallaro and Dejana, supra; Albergaria. A. et al, P-cadherin role in normal breast development and cancer. Int J Dev Biol. 2011; 55 (7-9):811-22).

P-cadherin (Placental-Cadherin, or Cadherin-3, encoded by the CDH3 gene in human) is a 118 kDa glycoprotein classic cadherin with a 26 amino acid long signal sequence and an 803 amino acid propeptide. The mature protein begins at 108 with three distinct domains: five extracellular cadherin repeats (548 aa), which is essential for the creation of lateral dimmers that act together in a zipper-like structure between neighboring cells; single transmembrane region (23 aa); highly conserved cytoplasmic tail (151 aa), an intracellular domain that interacts with catenins, which connect cadherins to the actin cytoskeleton.

P-cadherin is expressed in placenta of mice, also in human placental tissues (lower levels) and several human fetal structures. In adults, it is only expressed in certain tissues, usually co-expressed with E-cadherin, such as the basal layer of epidermis, breast, prostate, mesothelium, ovary, hair follicle, and corneal endothelium (Imai et al., Identification of a novel tumor-associated antigen, cadherin 3/P-cadherin, as a possible target for immunotherapy of pancreatic, gastric, and colorectal cancers. Clin. Cancer Res. 2008, 14, 6487-6495). Major sites of expression indicated by human protein reference database (HPRD: 00227) are endometrium, glomerulus, hair follicle, keratinocytes, mammary myoepithelium, melanocytes, oocytes, spermatozoa, placenta, prostate, retina, serum, and skin.

P-cadherin has been shown to be overexpressed in breast cancer and other tumors and may correlate with poor prognosis. It also showed high expression and positive rate in multiple cancers such as colorectal, NSCLC, gastric cancer and pancreatic cancers. In TCGA database, P-cadherin showed >5 fold higher expression in tumors of: cholangio (10.6×), colon (134× and 104×), esophageal (34×), lung (6.56× and 11.8×), stomach (8.02× and 11.6×) and thyroid (20.3×). P-cadherin may mediate tumor promoting effects including cell invasion, cell motility, stem cell activity and metastases formation in different tissue contexts. P-cadherin gene expression in normal tissues are very low, showing only very weak expression in ovary and mammary gland (GTex data base and literature). Pfizer's humanized monoclonal antibody anti-P-cadherin mAb PF-03732010 showed safety in Phase I trials however observed no clear beneficial effect in these patients.

In the present disclosure, monoclonal antibodies against P-cadherin which can be used to treat a variety of cancers have been developed.

### SUMMARY

These and other objectives are provided for by the present disclosure which, in a broad sense, is directed to compounds, methods, compositions and articles of manufacture that provide antibodies with improved efficacy. The benefits provided by the present disclosure are broadly applicable in the field of antibody therapeutics and diagnostics and may be used in conjunction with antibodies that react with a variety of targets.

The present disclosure provides antibodies against P-cadherin, nucleic acid molecules encoding the anti-P-cadherin antibodies, expression vectors and host cells used for the expression of anti-P-cadherin antibodies, and methods for validating the function of antibodies in vitro. The antibodies of the present disclosure provide a very potent agent for the treatment of multiple cancers via modulating human immune function.

In some aspects, the present disclosure comprises an isolated antibody, or an antigen-binding portion thereof against P-cadherin, such as human P-cadherin, mouse P-cadherin or cynomolgus monkey P-cadherin.

In some embodiments, the isolated antibody or antigen-binding portion thereof comprises:
(A) one or more heavy chain CDRs (HCDRs) selected from the group consisting of:
   (i) a HCDR1 comprising SEQ ID NO: 2;
   (ii) a HCDR2 comprising SEQ ID NO: 4; and
   (iii) a HCDR3 comprising SEQ ID NO: 6, 8, 9, 10 or 11;
(B) one or more light chain CDRs (LCDRs) selected from the group consisting of:
   (i) a LCDR1 comprising SEQ ID NO: 13;
   (ii) a LCDR2 comprising SEQ ID NO: 15; and
   (iii) a LCDR3 comprising SEQ ID NO: 17; or
(C) one or more HCDRs of (A) and one or more LCDRs of (B).

In some embodiments, the isolated antibody or antigen-binding portion thereof that specifically binds to P-cadherin comprises:
a HCDR1 comprising SEQ ID NO: 2 or an amino acid sequence that differs from SEQ ID No: 2 by an amino acid substitution, addition and/or deletion of not more than 2 amino acids;
a HCDR2 comprising SEQ ID NO: 4 or an amino acid sequence that differs from SEQ ID No: 4 by an amino acid substitution, addition and/or deletion of not more than 2 amino acids;
a HCDR3 comprising SEQ ID NO: 6 or an amino acid sequence that differs from SEQ ID No: 6 by an amino acid substitution, addition and/or deletion of not more than 2 amino acids;
a LCDR1 comprising SEQ ID NO: 13 or an amino acid sequence that differs from SEQ ID No: 13 by an amino acid substitution, addition and/or deletion of not more than 2 amino acids;
a LCDR2 comprising SEQ ID NO: 15 or an amino acid sequence that differs from SEQ ID No: 15 by an amino acid substitution, addition and/or deletion of not more than 2 amino acids; and
a LCDR3 comprising SEQ ID NO: 17 or an amino acid sequence that differs from SEQ ID No: 17 by an amino acid substitution, addition and/or deletion of not more than 2 amino acids.

In some embodiments, the isolated antibody or antigen-binding portion thereof have essentially the same CDRs as those of W3195-1.53.1-uIgG1L, except that PTM removal are performed on the CDRs to avoid the potential risk of post translational modification.

In some embodiments, the isolated antibody or antigen-binding portion thereof that specifically binds to P-cadherin comprises:
a HCDR1 comprising SEQ ID NO: 2;
a HCDR2 comprising SEQ ID NO: 4;
a HCDR3 comprising SEQ ID NO: 6 or an amino acid sequence that differs from SEQ ID No: 6 by an amino acid substitution, addition and/or deletion of not more than 2 amino acids (e.g. not more than one amino acid);
a LCDR1 comprising SEQ ID NO: 13;
a LCDR2 comprising SEQ ID NO: 15; and
a LCDR3 comprising SEQ ID NO: 17.

In some embodiments, the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 6; and
(B) a LCDR1 as set forth in SEQ ID NO: 13; a LCDR2 as set forth in SEQ ID NO: 15; and a LCDR3 as set forth in SEQ ID NO: 17.

In some embodiments, the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 8; and
(B) a LCDR1 as set forth in SEQ ID NO: 13; a LCDR2 as set forth in SEQ ID NO: 15; and a LCDR3 as set forth in SEQ ID NO: 17.

In some embodiments, the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 9; and
(B) a LCDR1 as set forth in SEQ ID NO: 13; a LCDR2 as set forth in SEQ ID NO: 15; and a LCDR3 as set forth in SEQ ID NO: 17.

In some embodiments, the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 10; and
(B) a LCDR1 as set forth in SEQ ID NO: 13; a LCDR2 as set forth in SEQ ID NO: 15; and a LCDR3 as set forth in SEQ ID NO: 17.

In some embodiments, the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 11; and
(B) a LCDR1 as set forth in SEQ ID NO: 13; a LCDR2 as set forth in SEQ ID NO: 15; and a LCDR3 as set forth in SEQ ID NO: 17.

In some embodiments, the isolated antibody or antigen-binding portion thereof comprises a heavy chain variable region (VH) and a light chain variable region (VL), wherein
the VH comprises one or more heavy chain CDRs (HCDRs) selected from the group consisting of:
   (i) a HCDR1 comprising SEQ ID NO: 2;
   (ii) a HCDR2 comprising SEQ ID NO: 4; and
   (iii) a HCDR3 comprising SEQ ID NO: 6, 8, 9, 10 or 11; and
the VL comprises one or more light chain CDRs (LCDRs) selected from the group consisting of:
   (i) a LCDR1 comprising SEQ ID NO: 13;
   (ii) a LCDR2 comprising SEQ ID NO: 15; and
   (iii) a LCDR3 comprising SEQ ID NO: 17.

In some embodiments, the isolated antibody or antigen-binding portion thereof comprises:
(A) a heavy chain variable region (VH):
   (i) comprising the amino acid sequence as set forth in any of SEQ ID NOs: 21-25;
   (ii) comprising an amino acid sequence at least 85%, 90%, or 95% identical to the amino acid sequence as set forth in any of SEQ ID NOs: 21-25 yet retaining the specific binding affinity to P-cadherin; or
   (iii) comprising an amino acid sequence with addition, deletion and/or substitution of one or more (e.g. two or three) amino acids compared with the amino acid sequence as set forth in any of SEQ ID NOs: 21-25; and/or
(B) a light chain variable region (VL):
   (i) comprising the amino acid sequence as set forth in any of SEQ ID NOs: 26-28;
   (ii) comprising an amino acid sequence at least 85%, at least 90%, or at least 95% identical to the amino acid sequence as set forth in any of SEQ ID NOs: 26-28 yet retaining the specific binding affinity to P-cadherin; or
   (iii) comprising an amino acid sequence with addition, deletion and/or substitution of one or more (e.g. two or three) amino acids compared with the amino acid sequence as set forth in any of SEQ ID NOs: 26-28.

In some embodiments, the addition, deletion and/or substitution of at least one of the amino acids in the VH or VL region is not in any of the CDR sequences, but in the framework (FRW) sequences.

In some embodiments, the isolated antibody or antigen-binding portion thereof as described above further comprises one or more substitutions of the amino acids in the framework sequences, e.g. FRW1, FRW2, FRW3, and/or FRW4 of the VH or VL region. The isolated antibody or antigen-binding portion thereof may have essentially the same framework sequences as those of W3195-1.53.1-uIgG1L, except that PTM removal are performed on the framework sequences to avoid the potential risk of post translational modification.

In some embodiments, the isolated antibody or antigen-binding portion thereof comprises a heavy chain variable region (VH) and a light chain variable region (VL), wherein
the VH comprises one or more heavy chain FRWs (HFRWs) selected from the group consisting of:
   (i) a HFRW1 comprising SEQ ID NO: 1;
   (ii) a HFRW2 comprising SEQ ID NO: 3;
   (iii) a HFRW3 comprising SEQ ID NO: 5; and
   (iv) a HFRW4 comprising SEQ ID NO: 7; and
the VL comprises one or more light chain FRWs (LFRWs) selected from the group consisting of:
   (i) a LFRW1 comprising SEQ ID NO: 12, 19 or 20;
   (ii) a LFRW2 comprising SEQ ID NO: 14;
   (iii) a LFRW3 comprising SEQ ID NO: 16; and
   (iv) a LFRW4 comprising SEQ ID NO: 18.

In some embodiments, the isolated antibody or antigen-binding portion thereof comprises a heavy chain variable region comprising the amino acid sequence as set forth in SEQ ID NO: 21, 22, 23, 24 or 25; and a light chain variable region comprising the amino acid sequence as set forth in SEQ ID NO: 26, 27 or 28.

In some embodiments, the isolated antibody or antigen-binding portion thereof comprises a heavy chain variable region as set forth in SEQ ID NO: 21 and a light chain variable region as set forth in SEQ ID NO: 26.

In some embodiments, the isolated antibody or antigen-binding portion thereof comprises a heavy chain variable region as set forth in SEQ ID NO: 21 and a light chain variable region as set forth in SEQ ID NO: 27.

In some embodiments, the isolated antibody or antigen-binding portion thereof comprises a heavy chain variable region as set forth in SEQ ID NO: 21 and a light chain variable region as set forth in SEQ ID NO: 28.

In some embodiments, the isolated antibody or antigen-binding portion thereof comprises a heavy chain variable region as set forth in SEQ ID NO: 22 and a light chain variable region as set forth in SEQ ID NO: 27.

In some embodiments, the isolated antibody or antigen-binding portion thereof comprises a heavy chain variable region as set forth in SEQ ID NO: 23 and a light chain variable region as set forth in SEQ ID NO: 27.

In some embodiments, the isolated antibody or antigen-binding portion thereof comprises a heavy chain variable region as set forth in SEQ ID NO: 24 and a light chain variable region as set forth in SEQ ID NO: 27.

In some embodiments, the isolated antibody or antigen-binding portion thereof comprises a heavy chain variable region as set forth in SEQ ID NO: 25 and a light chain variable region as set forth in SEQ ID NO: 27.

In some embodiments, the isolated antibody or antigen-binding portion thereof as disclosed herein further comprises a human IgG constant domain, such as a human IgG1, IgG2, IgG3 or IgG4 constant domain, optionally a human IgG1 constant domain. In some further embodiments, the isolated antibody or antigen-binding portion thereof comprises a human IgG1 Fc variant, e.g. an IgG1 Fc with L234A/L235A substitutions, according to EU numbering. Specifically, the heavy chain constant domain may comprise the amino acid sequence as set forth in SEQ ID NO: 29 or SEQ ID NO: 31.

In some embodiments, the isolated antibody or the antigen-binding portion thereof as disclosed herein has one or more of the following properties:
(a) bind to cell surface human P-cadherin or cynomolgus monkey P-cadherin with an EC50 in nM grade (e.g. no more than 1 nM, no more than 0.5 nM, no more than 0.3 nM, no more than 0.2 nM, or no more than 0.1 nM), as measured by FACS;
(b) bind to cell surface human P-cadherin with a KD no more than 0.1 nM (e.g. no more than 0.08 nM, no more than 0.05 nM, no more than 0.04 nM, or no more than 0.03 nM), as measured by FACS affinity test;
(c) have no cross-reactivity to human E-cadherin or N-cadherin;
(d) have good internalization ability comparable with benchmark antibodies;
(e) have significantly better ADCC effect than benchmark antibodies;
(f) inhibit the aggregation of human P-cadherin expressing cells with an EC50 in nM grade;
(g) show no non-specific binding effect; and
(h) being stable in serum for at least 14 days.

In some embodiments, the isolated antibody or antigen-binding portion thereof as disclosed herein is a chimeric antibody, a humanized antibody or a fully human antibody. Preferably, the antibody is a fully human monoclonal antibody.

In some embodiments, the isolated antibody or antigen-binding portion thereof as disclosed herein comprises a heavy chain and a light chain, wherein:
(a) the heavy chain comprises a heavy chain variable region as set forth in SEQ ID NO: 21, 22, 23, 24 or 25, and a heavy chain constant region as set forth in SEQ ID NO: 29 or 31; and
(b) the light chain comprises a light chain variable region as set forth in SEQ ID NO: 26, 27 or 28, and a light chain constant region as set forth in SEQ ID NO: 30.

In some embodiments, the isolated antibody or antigen-binding portion thereof as disclosed herein comprises a heavy chain and a light chain, wherein:
(a) the heavy chain comprises a heavy chain variable region as set forth in SEQ ID NO: 24, and a heavy chain constant region as set forth in SEQ ID NO: 31; and
(b) the light chain comprises a light chain variable region as set forth in SEQ ID NO: 27, and a light chain constant region as set forth in SEQ ID NO: 30.

In some aspects, the present disclosure is directed to an isolated nucleic acid molecule, comprising a nucleic acid sequence encoding the heavy chain variable region and/or the light chain variable region of the isolated antibody as disclosed herein.

In some aspects, the present disclosure is directed to a vector comprising the nucleic acid molecule encoding the antibody or antigen-binding portion thereof as disclosed herein.

In some aspects, the present disclosure is directed to a host cell comprising the expression vector as disclosed herein.

In some aspects, the present disclosure is directed to a pharmaceutical composition comprising at least one antibody or antigen-binding portion thereof as disclosed herein and a pharmaceutically acceptable carrier.

In some aspects, the present disclosure is directed to a method for preparing an anti-P-cadherin antibody or antigen-binding portion thereof which comprises expressing the antibody or antigen-binding portion thereof in the host cell as disclosed above and isolating the antibody or antigen-binding portion thereof from the host cell.

In some aspects, the present disclosure is directed to a method of modulating an P-cadherin-related immune response in a subject, comprising administering the antibody or antigen-binding portion thereof as disclosed herein to the subject such that the P-cadherin-related immune response in the subject is modulated.

In some aspects, the present disclosure is directed to a method for treating or preventing a P-cadherin positive cancer in a subject, comprising administering an effective amount of the antibody or antigen-binding portion thereof or the pharmaceutical composition as disclosed herein to the subject. In some embodiments, said cancer is P-Cadherin positive solid tumor. In some embodiments, said cancer can be selected from cholangio carcinoma, esophageal cancer, oral cancers, thyroid tumor, head and neck cancer, breast cancer, lung cancer, NSCLC, SCLC, malignant mesothelioma, colon cancer, colorectal cancer, ovarian cancer, cervical cancer, melanoma, skin cancer, bladder cancer, liver cancer, prostate cancer, stomach cancer, kidney cancers, pancreatic cancer, endometrial cancer, urothelial cancer, sarcoma, osteosarcoma, and bone cancer.

In some aspects, the present disclosure is directed to the use of the antibody or antigen-binding portion thereof as disclosed herein in the manufacture of a medicament for diagnosing, treating or preventing P-cadherin positive cancer.

In some aspects, the present disclosure is directed to the antibody or antigen-binding portion thereof as disclosed herein for use in diagnosing, treating or preventing P-cadherin positive cancer.

In some aspects, the present disclosure is directed to kits or devices and associated methods that employ the antibody or antigen-binding portion thereof as disclosed herein, and pharmaceutical compositions as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1A shows the serum titer data on human P-cadherin (hPro1.ECD.his) in immunized OMT rats, measured by ELISA.

FIGS. 1B and 1C show the SDS-PAGE and Size exclusion chromatography (SEC-HPLC) result of the antibody, respectively.

FIG. 2 shows the affinity result of antibodies on human P-cadherin expressing DU-145 cells, as tested by FACS affinity analysis.

FIG. 3 shows the result of FACS binding assay of antibodies on human P-cadherin expressing DU-145 (FIG. 3A), NCI-H1650 (FIG. 3B) and HCT116 (FIG. 3C) cells, respectively.

FIG. 4 shows the result of FACS binding assay of antibodies on cyno P-Cadherin transiently expressing W319-CHOK1.cynoPro1.FL cells.

FIG. 5 shows the binding result of antibodies to human E-Cadherin (FIG. 5A, WBP319-hPro2.ECD.His) and human N-Cadherin (FIG. 5B, WBP319-hPro3.ECD.hFcHis), measured by ELISA.

FIG. 6 shows the result of domain determination binding of antibodies, measured by ELISA. The tested regions are human P-cadherin Extracellular domain 1 (FIG. 6A, aa 108-236, W319-hPro1.D1.ECD.hFc), domain 1-2 (FIG. 6B, aa 108-348, W319-hPro1.D12.ECD.hFc (WT)), domain 1-3 (FIG. 6C, aa 108-461, W319-hPro1.D123.ECD.hFc (WT)), domain 1-4 (FIG. 6D, aa 108-550, W319-hPro1.D1234.ECD.hFc (WT)) and W319-hPro1.ECD.hFc (WT)-P3 (FIG. 6E). W319-hPro1.ECD.hFc (WT)-P3 refers to a fraction of the human P-cadherin Extracellular protein flow during purification.

FIG. 7 shows the internalization ability of antibodies by Fab-ZAP CTG assay (FIG. 7A) and HCS assay (FIG. 7B).

FIG. 8 shows the ADCC effect of antibodies on human P-cadherin expressing HCT-116 cells using reporter gene assay (RGA).

FIG. 9A-9B show the ability of antibodies to interfere with P-cadherin-dependent cell aggregation by cell aggregation assay.

FIG. 10 shows the serum stability result of W3195-1.53.1-p1-uIgG1L by comparable FACS binding.

FIGS. 11 shows the comparison of different antibodies in (FIG. 11A) FACS binding test on HCT-116 cells; (FIG. 11B) FACS binding test on DU-145 cells; and (FIG. 11C) internalization test on HCC-1954 cells by Fab-ZAP.

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “antibody” or “Ab,” as used herein, generally refers to a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, δ, γ, α and ε, which define isotypes of an antibody as IgM, IgD, IgG, IgA and IgE, respectively. In a light chain and a heavy chain, a variable region is linked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. The extent of the framework region and CDRs can be precisely identified using methodology known in the art, for example, by the Kabat definition, the Chothia definition, the AbM definition, the EU definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Edelman et al., Proc Natl Acad Sci USA. 1969 May; 63(1):78-85; and Almagro, J. Mol. Recognit. 17:132-143 (2004). See also hgmp.mrc.ac.uk and bioinforg.uk/abs. Correspondence or alignments between numberings according to different definitions can for example be found at http:www.imgt.org/ (see also Giudicelli V et al. IMGT, the international ImMunoGeneTics database. Nucleic Acids Res. (1997) 25:206-11; Lefranc M P et al. Unique database numbering system for immunogenetic analysis. Immunol Today (1997) 18:509; and Lefranc M P et al., IMGT unique numbering for immunoglobulin and T cell receptor variable domains and Ig superfamily V-like domains. Dev Comp Immunol. (2003) 27:55-77). Antibodies may be of different antibody isotypes, for example, IgG (e.g., IgG1, IgG2, IgG3 or IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, and/or compete with the full-length antibody for binding to the same antigen. Generally, see Fundamental Immunology, Ch. 7 (Paul, W., ed., the second edition, Raven Press, N.Y. (1989), which is incorporated herein by reference for all purposes. Antigen binding fragments of an antibody may be produced by recombinant DNA techniques or by enzymatic or chemical cleavage of an intact antibody. Under some conditions, antigen binding fragments include Fab, Fab′, F(ab′)₂, Fd, Fv, dAb and complementary determining region (CDR) fragments, single chain antibody (e.g. scFv), chimeric antibody, diabody and such polypeptides that comprise at least part of antibody sufficient to confer the specific antigen binding ability on the polypeptides. Antigen binding fragments of an antibody may be obtained from a given antibody (e.g., the monoclonal anti-human P-cadherin antibody provided in the instant application) by conventional techniques known by a person skilled in the art (e.g., recombinant DNA technique or enzymatic or chemical cleavage methods), and may be screened for specificity in the same manner by which intact antibodies are screened.

“Framework regions” (or FRWs), as used herein, refer to those variable domain residues other than the CDR residues. Each variable domain typically has four FRs identified as FRW1, FRW2, FRW3 and FRW4.

“Fc” with regard to an antibody refers to that portion of the antibody comprising the second and third constant regions of a first heavy chain bound to the second and third constant regions of a second heavy chain via disulfide bonding, optionally also comprising part or whole of the hinge region. The Fc portion of the antibody is responsible for various effector functions such as antibody-dependent cell-mediated cytotoxicity (ADCC), and complement dependent cytotoxicity (CDC), but does not function in antigen binding.

The term “monoclonal antibody” or “mAb,” as used herein, refer to a preparation of antibody molecules of single molecular composition. A monoclonal antibody displays a binding specificity and affinity for a particular antigen.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “recombinant antibody,” as used herein, refers to an antibody that is prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “fully human” as used herein, with reference to antibody or antigen-binding domain, means that the antibody or the antigen-binding domain has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

The term “P-cadherin”, as used herein, refers to placental Cadherin (despite its name, P-cadherin is not expressed in human placenta; its name results from the fact that this molecule was originally observed to be highly expressed in mouse placenta throughout pregnancy) and is a member of the classical cadherin family of transmembrane glycoproteins that regulate cell-cell adhesion. The sequences of human P-cadherin (encoded by CDH3 gene) can be obtained from Uniprot database under ID P22223, including a canonical sequence and several isoforms. The term “P-cadherin” is intended to include recombinant human, mouse, cyno P-cadherin and recombinant chimeric forms of P-cadherin, which can be prepared by standard recombinant expression methods or purchased commercially. The canonical P-cadherin sequence comprises 829 amino acids, wherein the mature protein begins at amino acid 108 with three distinct domains: five extracellular cadherin repeats (548 aa), single transmembrane region (23 aa) and highly conserved cytoplasmic tail (151 aa).

The terms “E-cadherin” and “N-cadherin”, as used herein, refers to epithelial Cadherin and neural cadherin, respectively, which are also members of the classical cadherin family. Cadherins are divided into type I and type II subgroups. Type I cadherins include E-cadherin, N-cadherin, P-cadherin and retinal cadherin (R-cadherin), whereas kidney cadherin (K-cadherin) and osteoblast cadherin (OB-cadherin) are type II cadherins. E-cadherin is encoded by CDH1 gene in human, which shares 66% homology with CDH3 gene. N-cadherin is encoded by CDH2 gene in human. E-cadherin, N-cadherin and P-cadherin are the best characterized subgroup of adhesion proteins.

The term “anti-P-cadherin antibody” or “P-cadherin antibody” or “antibody against P-cadherin,” as used herein, refers to an antibody, as defined herein, capable of binding to a P-cadherin, for example, binding to the ECD region of a human P-cadherin protein.

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻¹⁰ M or less, more preferably 5×10⁻¹¹ M or less, even more preferably 4×10⁻¹¹ M or less for a target antigen, for example, P-cadherin, as measured by FACS affinity test.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds a P-cadherin protein is substantially free of antibodies that specifically bind antigens other than P-cadherin proteins). An isolated antibody that specifically binds a human P-cadherin protein may, however, have cross- reactivity to other antigens, such as P-cadherin proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human P-cadherin gene was transfected into 293F cells.

The term “hybridoma” and the term “hybridoma cell line,” as used herein, may be used interchangeably. When the term “hybridoma” and the term “hybridoma cell line” are mentioned, they also include subclone and progeny cell of hybridoma.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of P-cadherin-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### Anti-P-cadherin Antibodies

In some aspects, the disclosure comprises an isolated antibody or an antigen-binding portion thereof against P-cadherin.

In the context of the application, the “antibody” may include polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof; and derivatives thereof including Fc fusions and other modifications, and any other immune-reactive molecule so long as it exhibits preferential association or binding with a P-cadherin protein. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). In a preferred embodiment, the antibody is a monoclonal antibody. In a more preferred embodiment, the antibody is a humanized monoclonal antibody or fully human monoclonal antibody.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and art-recognized biochemical and genetic engineering techniques such as described in more detail in An, Zhigiang (ed.) Therapeutic Monoclonal Antibodies: From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981) each of which is incorporated herein in its entirety by reference. In some embodiments, the antibody as disclosed herein is obtained by utilizing hybridoma technology and genetically engineered OmniRat (developed by Open Monoclonal Technology (OMT) Company). It should be understood that a selected binding sequence can be further altered, for example, to improve affinity for the target, to humanize the target binding sequence, to improve its production in cell culture, to reduce its immunogenicity in vivo, to create a multi-specific antibody, etc., and that an antibody comprising the altered target binding sequence is also an antibody of this disclosure.

In a preferred embodiment, the anti-human P-cadherin monoclonal antibody is prepared by using hybridoma techniques. Generation of hybridomas is well-known in the art. See, e.g., Harlow and Lane (1988) Antibodies, A Laboratory Manual, Cold Spring Harbor Publications, New York.

In this application, a series of screening processes is performed for identifying the positive hybridoma cell lines. The goal of the screening process is to find candidate human P-cadherin high affinity binders for constructing the antibody. The sequences of the antibodies are further optimized (e.g. PTM removal and/or Fc modification) to obtain an antibody with high binding affinity and suitable functional activities.

The antibody of the disclosure can bind to both human and cynomolgus monkey P-cadherin with high affinity. The binding of an antibody of the disclosure to P-cadherin can be assessed using one or more techniques well established in the art, for instance, ELISA. The binding specificity of an antibody of the disclosure can also be determined by monitoring binding of the antibody to cells expressing a P-cadherin protein, e.g., flow cytometry. For example, an antibody can be tested by a flow cytometry assay in which the antibody is reacted with a cell line that expresses human P-cadherin, such as CHO K1 cells that have been transfected to express P-cadherin on their cell surface. Additionally, or alternatively, the binding of the antibody, including the binding kinetics (e.g., K_{d} value) can be tested in BIAcore binding assays. Still other suitable binding assays include ELISA assays, for example using a recombinant P-cadherin protein. For instance, an antibody of the disclosure binds to a human P-cadherin with a KD of 1×10⁻⁹ M or less, binds to a human P-cadherin with a KD of 5×10⁻¹⁰ M or less, binds to a human P-cadherin with a KD of 2×10⁻¹⁰ M or less, binds to a human P-cadherin protein with a KD of 1×10⁻¹⁰ M or less, binds to a human P-cadherin protein with a KD of 5×10 -11 M or less, binds to a human P-cadherin protein with a KD of 3×10⁻¹¹ M or less, or binds to a human P-cadherin protein with a KD of 2×10⁻¹¹ M or less, as measured by FACS affinity test.

### Anti-P-Cadherin Antibodies Comprising CDRs and Framework Sequences

In some aspects, the isolated antibody or antigen-binding portion thereof as disclosed herein comprises:
(A) one or more heavy chain CDRs (HCDRs) selected from the group consisting of:
   (i) a HCDR1 comprising SEQ ID NO: 2;
   (ii) a HCDR2 comprising SEQ ID NO: 4; and
   (iii) a HCDR3 comprising SEQ ID NO: 6 or a variant thereof (such as SEQ ID NO: 8, 9, 10, 11);
(B) one or more light chain CDRs (LCDRs) selected from the group consisting of:
   (i) a LCDR1 comprising SEQ ID NO: 13;
   (ii) a LCDR2 comprising SEQ ID NO: 15; and
   (iii) a LCDR3 comprising SEQ ID NO: 17; or
(C) one or more HCDRs of (A) and one or more LCDRs of (B).

Variable regions and CDRs in an antibody sequence can be identified according to general rules that have been developed in the art (as set out above, such as the Kabat, Chothia and IMGT numbering system) or by aligning the sequences against a database of known variable regions. Methods for identifying these regions are described in Kontermann and Dubel, eds., Antibody Engineering, Springer, New York, NY, 2001 and Dinarello et al., Current Protocols in Immunology, John Wiley and Sons Inc., Hoboken, NJ, 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671 -D674 (2005). For example, sequences may be analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Two antibodies having the same VH and/or VL CDRs means that their CDRs are identical when determined by the same approach (e.g., the Kabat approach, the Chothia approach, or the IMGT numbering as known in the art). The CDRs set forth herein are generally derived according to a union of Kabat and IMGT numbering.

CDRs are known to be responsible for antigen binding, however, it has been found that not all of the 6 CDRs are indispensable or unchangeable. In other words, it is possible to replace or change or modify one or more CDRs provided herein, yet substantially retain the specific binding affinity to P-cadherin.

Further, heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the variant of SEQ ID NO: 6 comprised in HCDR3 differs from SEQ ID NO: 6 by an addition, deletion and/or substitution of no more than two amino acids, preferably one amino acid, and preferably is a substitution. For example, the variants of SEQ ID NO: 6 comprise an amino acid sequence that differs from SEQ ID No: 6 by an amino acid addition, deletion or substitution of not more than 2 amino acids. In some embodiments, the variants of SEQ ID NO: 6 are those as set forth in SEQ ID NO: 8, 9, 10 and 11, but are not limited to these.

In some specific embodiments, the HCDR3 as described above may comprise or consist of the sequence as set forth in SEQ ID NO: 6. In some embodiments, such antibodies include W3195-1.53.1-uIgG1L (i.e. the parental antibody), W3195-1.53.1-p1-uIgG1L (or referred to as W3195-p1 antibody), and W3195-1.53.1-p3-uIgG1L (or referred to as W3195-p3 antibody) as disclosed herein.

In some specific embodiments, the HCDR3 as described above may comprise or consist of the sequence as set forth in SEQ ID NO: 8. In some embodiments, such antibodies include W3195-1.53.1-p4-uIgG1V320 (or referred to as W3195-p4-V320 antibody) as disclosed herein. The suffix “V320” means that the IgG1 Fc region comprises a L234A/L235A substitution.

In some specific embodiments, the HCDR3 as described above may comprise or consist of the sequence as set forth in SEQ ID NO: 9. In some embodiments, such antibodies include W3195-1.53.1-p4-uIgG1V320 (or referred to as W3195-p5-V320 antibody) as disclosed herein.

In some specific embodiments, the HCDR3 as described above may comprise or consist of the sequence as set forth in SEQ ID NO: 10. In some embodiments, such antibodies include W3195-1.53.1-p4-uIgG1V320 (or referred to as W3195-p6-V320 antibody) as disclosed herein.

In some specific embodiments, the HCDR3 as described above may comprise or consist of the sequence as set forth in SEQ ID NO: 11. In some embodiments, such antibodies include W3195-1.53.1-p4-uIgG1V320 (or referred to as W3195-p7-V320 antibody) as disclosed herein.

In some embodiments, the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 6 or 10; and
(B) a LCDR1 as set forth in SEQ ID NO: 13; a LCDR2 as set forth in SEQ ID NO: 15; and a LCDR3 as set forth in SEQ ID NO: 17.

### Anti-P-Cadherin Antibodies Comprising a Heavy Chain Variable Region and a Light Chain Variable Region

In some embodiments, the isolated antibody or antigen-binding portion thereof comprises a heavy chain variable region (VH) and a light chain variable region (VL), wherein:
the VH comprises one or more heavy chain CDRs (HCDRs) selected from the group consisting of:
   (i) a HCDR1 comprising SEQ ID NO: 2;
   (ii) a HCDR2 comprising SEQ ID NO: 4; and
   (iii) a HCDR3 comprising SEQ ID NO: 6, 8, 9, 10 or 11; and
the VL comprises one or more light chain CDRs (LCDRs) selected from the group consisting of:
   (i) a LCDR1 comprising SEQ ID NO: 13;
   (ii) a LCDR2 comprising SEQ ID NO: 15; and
   (iii) a LCDR3 comprising SEQ ID NO: 17.

In some embodiments, the isolated antibody or antigen-binding portion thereof comprises:
(A) a heavy chain variable region (VH):
   (i) comprising the amino acid sequence as set forth in SEQ ID NO: 21;
   (ii) comprising an amino acid sequence at least 85%, 90%, or 95% identical to the amino acid sequence as set forth in SEQ ID NO: 21 yet retaining the specific binding affinity to P-cadherin combined with the VL region; or
   (iii) comprising an amino acid sequence with addition, deletion and/or substitution of one or more (e.g. two or three) amino acids compared with the amino acid sequence as set forth in SEQ ID NO: 21; and/or
(B) a light chain variable region (VL):
   (i) comprising the amino acid sequence as set forth in SEQ ID NO: 26;
   (ii) comprising an amino acid sequence at least 85%, at least 90%, or at least 95% identical to the amino acid sequence as set forth in SEQ ID NO: 26 yet retaining the specific binding affinity to P-cadherin combined with the VL region; or
   (iii) comprising an amino acid sequence with addition, deletion and/or substitution of one or more (e.g. two or three) amino acids compared with the amino acid sequence as set forth in SEQ ID NO: 26.

In some embodiments, the isolated antibody or antigen-binding portion thereof comprises:
(A) a heavy chain variable region (VH):
   (i) comprising the amino acid sequence as set forth in SEQ ID NO: 24;
   (ii) comprising an amino acid sequence at least 85%, 90%, or 95% identical to the amino acid sequence as set forth in SEQ ID NO: 24 yet retaining the specific binding affinity to P-cadherin combined with the VL region; or
   (iii) comprising an amino acid sequence with addition, deletion and/or substitution of one or more (e.g. two or three) amino acids compared with the amino acid sequence as set forth in SEQ ID NO: 24; and/or
(B) a light chain variable region (VL):
   (i) comprising the amino acid sequence as set forth in SEQ ID NO: 27;
   (ii) comprising an amino acid sequence at least 85%, at least 90%, or at least 95% identical to the amino acid sequence as set forth in SEQ ID NO: 27 yet retaining the specific binding affinity to P-cadherin combined with the VL region; or
   (iii) comprising an amino acid sequence with addition, deletion and/or substitution of one or more (e.g. two or three) amino acids compared with the amino acid sequence as set forth in SEQ ID NO: 27.

In some specific embodiments, the isolated antibody or antigen-binding portion thereof comprises: a heavy chain variable region comprising or consisting of the amino acid sequence of SEQ ID NO: 21 or 24 and a light chain variable region comprising or consisting of the amino acid sequence of SEQ ID NO: 26 or 27.

In other embodiments, the amino acid sequences of the heavy chain variable region and/or the light chain variable region can be at least 85%, 86%, 87%, 88%, 89%, 90%, 91%, 92%, 93%, 94%, 95%, 96%, 97%, 98% or 99% identical to the respective sequences set forth above.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. MoI. Biol. 215:403-10. BLAST protein searches can be performed with the XBLAST program, score=50, wordlength=3 to obtain amino acid sequences homologous to the antibody molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In some embodiments, the addition, deletion and/or substitution of at least one of the amino acids in the VH or VL region is not in any of the CDR sequences, but in the framework (FRW) sequences. For example, the isolated antibody or antigen-binding portion thereof as described above may comprise one or more substitutions of the amino acids in the framework sequences, e.g. FRW1, FRW2, FRW3, and/or FRW4 of the VH or VL region.

In certain embodiments, the isolated antibody or antigen-binding portion thereof as provided herein comprise any suitable framework region (FR) sequences, as long as the antigen-binding domains can specifically bind to P-cadherin.

In some embodiments, the isolated antibody or antigen-binding portion thereof comprises a heavy chain variable region (VH) and a light chain variable region (VL), wherein:
the VH comprises one or more heavy chain FRWs (HFRWs) selected from the group consisting of:
   (i) a HFRW1 comprising SEQ ID NO: 1 or an amino acid sequence with an addition, deletion and/or substitution of one or more (e.g. 1, 2 or 3) amino acids compared to SEQ ID NO: 1;
   (ii) a HFRW2 comprising SEQ ID NO: 3 or an amino acid sequence with an addition, deletion and/or substitution of one or more (e.g. 1, 2 or 3) amino acids compared to SEQ ID NO: 3;
   (iii) a HFRW3 comprising SEQ ID NO: 5 or an amino acid sequence with an addition, deletion and/or substitution of one or more (e.g. 1, 2 or 3) amino acids compared to SEQ ID NO: 5; and
   (iv) a HFRW4 comprising SEQ ID NO: 7 or an amino acid sequence with an addition, deletion and/or substitution of one or more (e.g. 1, 2 or 3) amino acids compared to SEQ ID NO: 7; and
the VL comprises one or more light chain FRWs (LFRWs) selected from the group consisting of:
   (i) a LFRW1 comprising SEQ ID NO: 12, 19 or 20 or an amino acid sequence with an addition, deletion and/or substitution of one or more (e.g. 1, 2 or 3) amino acids compared to SEQ ID NO: 12, 19 or 20;
   (ii) a LFRW2 comprising SEQ ID NO: 14 or an amino acid sequence with an addition, deletion and/or substitution of one or more (e.g. 1, 2 or 3) amino acids compared to SEQ ID NO: 14;
   (iii) a LFRW3 comprising SEQ ID NO: 16 or an amino acid sequence with an addition, deletion and/or substitution of one or more (e.g. 1, 2 or 3) amino acids compared to SEQ ID NO: 16; and
   (iv) a LFRW4 comprising SEQ ID NO: 18 or an amino acid sequence with an addition, deletion and/or substitution of one or more (e.g. 1, 2 or 3) amino acids compared to SEQ ID NO: 18.

In some specific embodiments, the isolated antibody or antigen-binding portion thereof comprises a heavy chain variable region (VH) and a light chain variable region (VL), wherein
the VH comprises one or more heavy chain FRWs (HFRWs) selected from the group consisting of:
   (i) a HFRW1 comprising SEQ ID NO: 1;
   (ii) a HFRW2 comprising SEQ ID NO: 3;
   (iii) a HFRW3 comprising SEQ ID NO: 5; and
   (iv) a HFRW4 comprising SEQ ID NO: 7; and
the VL comprises one or more light chain FRWs (LFRWs) selected from the group consisting of:
   (i) a LFRW1 comprising SEQ ID NO: 12, 19 or 20;
   (ii) a LFRW2 comprising SEQ ID NO: 14;
   (iii) a LFRW3 comprising SEQ ID NO: 16; and
   (iv) a LFRW4 comprising SEQ ID NO: 18.

“PTM sites” or “post-translational modification sites”, as used herein, refers to amino acids or motifs in antibody sequences that are prone to cause a variety of modifications, and thus affecting the biological activity and therapeutic effects of antibodies in vivo. PTMs vary from chain additions, such as N- and O-linked glycosylation, glycation, cysteinylation and sulfation; chain trimming, such as C-terminal lysine clipping; amino acid modifications such as cyclization (into a N-terminal pyroglutamic acid), deamidation, oxidation, isomerization and carbamylation; to disulfide scrambling of hinge region interchain disulfide bonds. Typical sites for PTMs are known in the art, such as DG for isomerization, NG for deamination, N*T/S (* stand for other amino acid except P or D) for glycosylation, and M or C for oxidation and so on. As exemplified herein, once the PTM sites are found in the antibody sequences, especially in the key regions like CDR3, PTM removal are performed to avoid the potential risk of PTM modification. PTM removal is often achieved by a conservative substitution.

As described above, the isolated antibody or antigen-binding portion thereof may contain modification of one or more amino acids in the variable regions of the heavy chain and/or light chain, preferably the modification is a conservative substitution. It is understood in the art that certain conservative sequence modification can be made which do not remove antigen binding. See, e.g., Brummell et al. (1993) Biochem 32:1180-8; de Wildt et al. (1997) Prot. Eng. 10:835-41; Komissarov et al. (1997) J. Biol. Chem. 272:26864- 26870; Hall et al. (1992) J. Immunol. 149:1605-12; Kelley and O'Connell (1993) Biochem. 32:6862-35; Adib-Conquy et al. (1998) Int. Immunol. 10:341-6 and Beers et al. (2000) Clin. Can. Res. 6:2835-43.

As described above, the term “conservative substitution,” as used herein, refers to amino acid substitutions which would not disadvantageously affect or change the essential properties of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, having similar size, shape, charge, chemical property including the capability of forming covalent bond or hydrogen bond, etc.) to the corresponding amino acid residue. The families of amino acid residues having similar side chains have been defined in the art. These families include amino acids having alkaline side chains (for example, lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, valine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32: 1180-1187 (1993); Kobayashi et al., Protein Eng. 12(10): 879-884 (1999); and Burks et al., Proc. Natl. Acad. Sci. USA 94: 412-417 (1997), which are incorporated herein by reference).

In some specific embodiments, the isolated antibody or antigen-binding portion thereof comprises a heavy chain variable region comprising the amino acid sequence as set forth in SEQ ID NO: 21, 22, 23, 24 or 25; and a light chain variable region comprising the amino acid sequence as set forth in SEQ ID NO: 26, 27 or 28.

The antigen-binding domain of the P-cadherin antibody comprising the VH and VL regions as described above may adopt a variety of formats, such as but not limited to, a Fab, a Fab′, a F(ab′)₂, an Fv fragment, a single-chain antibody molecule (scFv). In some embodiments, the antigen-binding domain is a Fv fragment consisted of the VH region and a VL region as described above in separate chains held together by tight, non-covalent interactions.

### IgG Constant Domain Comprising the Fc Region

The anti-P-cadherin antibodies and antigen-binding fragments provided herein further comprise a human IgG constant domain which comprises a Fc region and optionally a hinge region. The human IgG constant domain may be a human IgG1, IgG2, IgG3 or IgG4 constant domain, preferably a human IgG1 constant domain. In some embodiments, the Fc region is a human IgG1 Fc region. The Fc region may be, for example, a wild-type Fc region or comprising one or more amino acid modification (e.g. Leu234Ala/Leu235Ala or LALA) that alters the antibody-dependent cellular cytotoxicity (ADCC) or other effector functions.

In certain embodiments, the Fc modification comprise a LALA mutation, i.e. mutations of L234A and L235A, according to EU numbering as in Kabat et al. LALA mutation is perhaps the most commonly used mutation for disrupting antibody effector function, e.g. eliminate Fc binding to specific FcγRs, reduce ADCC activity mediated by PBMCs and monocytes. Non-limiting examples of Fc modifications also include, e.g., a mutation of serine (“S”) to proline (“P”) at position 228 of the amino acid sequence in case a human IgG4 Fc region is involved. The S228P mutation reduces Fab-arm exchange by stabilizing the disulfides in the core-hinge of the IgG4 molecules, thus belongs to an IgG4 stabilization mutation which helps prevent half-antibody formation.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU numbering as in Kabat” or “EU index as in Kabat” refers to the residue numbering of the human IgG1 EU antibody. Unless stated otherwise herein, references to residue numbers in the constant domain of antibodies means residue numbering by the EU numbering system.

### Anti-P-Cadherin Antibodies with Certain Properties

The antibodies of the present disclosure are characterized by particular functional features or properties of the antibodies. The in vitro functional characteristics and pharmacological activity of the antibodies have been fully assessed at the molecular and cellular levels according to the mechanism of action for the target. In some embodiments, the isolated antibodies or the antigen-binding portion thereof have one or more of the following properties:
(a) bind to cell surface human P-cadherin or cynomolgus monkey P-cadherin with an EC50 in nM grade (e.g. no more than 1 nM, no more than 0.5 nM, no more than 0.3 nM, no more than 0.2 nM, or no more than 0.1 nM), as measured by FACS, more specifically, can specifically bind to domain 3 (amino acids 349-461) of human P-cadherin ECD with an EC50 in nM grade (e.g. no more than 1 nM, no more than 0.5 nM, no more than 0.3 nM, no more than 0.2 nM, or no more than 0.1 nM);
(b) bind to cell surface human P-cadherin with a KD no more than 0.1 nM (e.g. no more than 0.08 nM, no more than 0.05 nM, no more than 0.04 nM, or no more than 0.03 nM), as measured by FACS affinity test;
(c) have no cross-reactivity to human E-cadherin or N-cadherin;
(d) have good internalization ability comparable with benchmark antibodies;
(e) have significantly better ADCC effect than benchmark antibodies;
(f) inhibit the aggregation of human P-cadherin expressing cells with an EC50 in nM grade;
   (g) show no non-specific binding effect;
(h) being stable in serum for at least 14 days; and
(i) compete for binding to P-cadherin (such as amino acids 349-461 of human P-cadherin) with an antibody selected from the group consisting of: W3195-1.53.1-uIgG1L, W3195-1.53.1-p1-uIgG1L, W3195-1.53.1-p3-uIgG1L, W3195-1.53.1-p4-uIgG1V320, W3195-1.53.1-p5-uIgG1V320, W3195-1.53.1-p6-uIgG1V320, and W3195-1.53.1-p7-uIgG1V320.

### Nucleic Acid Molecules Encoding Antibodies of the Disclosure

In some aspects, the disclosure is directed to an isolated nucleic acid molecule, comprising a nucleic acid sequence encoding the heavy chain variable region and/or the light chain variable region of the isolated antibody as disclosed herein.

Nucleic acids of the disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

The isolated nucleic acid encoding the VH region can be converted to a full-length heavy chain gene by operatively linking the VH-encoding nucleic acid to another DNA molecule encoding heavy chain constant regions (CH1, CH2 and CH3). The sequences of human heavy chain constant region genes are known in the art (see e.g., Kabat et al. (1991), supra) and DNA fragments encompassing these regions can be obtained by standard PCR amplification. The heavy chain constant region can be an IgG1, IgG2, IgG3, IgG4, IgA, IgE, IgM or IgD constant region, such as an IgG1 constant region.

The isolated nucleic acid encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat et al., supra) and DNA fragments encompassing these regions can be obtained by standard PCR amplification. In preferred embodiments, the light chain constant region can be a kappa or lambda constant region.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

In some embodiments, the disclosure is directed to an isolated nucleic acid molecule, comprising a nucleic acid sequence encoding the heavy chain variable region of the isolated antibody as disclosed herein.

In some specific embodiments, the isolated nucleic acid molecule encodes the heavy chain variable region of the isolated antibody and comprises a nucleic acid sequence selected from the group consisting of:
(A) a nucleic acid sequence that encodes a heavy chain variable region as set forth in SEQ ID NO: 21, 22, 23, 24 or 25;
(B) a nucleic acid sequence with at least 80% (e.g. at least 85%, 86%, 87%, 88%, 89%, 90%, 91%, 92%, 93%, 94%, 95%, 96%, 97%, 98%, or 99%) sequence identity to the nucleic acid sequence of (A); and
(C) a nucleic acid sequence that hybridizes under high stringency conditions to the complementary strand of the nucleic acid sequence of (A).

In some embodiments, the disclosure is directed to an isolated nucleic acid molecule, comprising a nucleic acid sequence encoding the light chain variable region of the isolated antibody as disclosed herein.

In some specific embodiments, the isolated nucleic acid molecule encodes the light chain variable region of the isolated antibody comprises a nucleic acid sequence selected from the group consisting of:
(A) a nucleic acid sequence that encodes a light chain variable region as set forth in SEQ ID NO: 26, 27, or 28;
(B) a nucleic acid sequence with at least 80% (e.g. at least 85%, 86%, 87%, 88%, 89%, 90%, 91%, 92%, 93%, 94%, 95%, 96%, 97%, 98%, or 99%) sequence identity to the nucleic acid sequence of (A); and
(C) a nucleic acid sequence that hybridized under high stringency conditions to the complementary strand of the nucleic acid sequence of (A).

In some specific embodiments, the percentage of identity is derived from the degeneracy of the genetic code, and the encoded protein sequences remain unchanged.

Exemplary high stringency conditions include hybridization at 45° C. in 5×SSPE and 45% formamide, and a final wash at 65° C. in 0.1×SSC. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al, (Eds.), Molecular Cloning: A laboratory Manual. Cold Spring Harbor Laboratory Press: Cold Spring Harbor, New York (1989), pp. 9.47 to 9.51.

### Host Cells

Host cells as disclosed in the present disclosure may be any cell which is suitable for expressing the antibodies of the present disclosure, for instance, bacterial, yeast, fungal, plant or animal cells, preferably mammalian cells. Mammalian host cells for expressing the antibodies of the present disclosure include Chinese Hamster Ovary (CHO cells) (including dhfr CHO cells, described in Urlaub and Chasin, (1980) Proc. Natl. Acad. ScL USA 77:4216-4220, used with a DHFR selectable marker, e.g., as described in R. J. Kaufman and P. A. Sharp (1982) J. MoI. Biol. 159:601-621), NSO myeloma cells, COS cells and SP2 cells. In particular, for use with NSO myeloma cells, another expression system is the GS gene expression system disclosed in WO 87/04462, WO 89/01036 and EP 338,841. When recombinant expression vectors encoding the antibody are introduced into mammalian host cells, the antibodies are produced by culturing the host cells for a period of time sufficient to allow for expression of the antibody in the host cells or, secretion of the antibody into the culture medium in which the host cells are grown. Antibodies can be recovered from the culture medium using standard protein purification methods.

### Pharmaceutical Compositions

In some aspects, the disclosure is directed to a pharmaceutical composition comprising at least one antibody or antigen-binding portion thereof as disclosed herein and a pharmaceutically acceptable carrier.

### Components of the Compositions

The pharmaceutical composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, an antiviral agent, or a vaccine, such that the anti-P-cadherin antibody enhances the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, in a solvent containing an antibody or an antigen-binding fragment of the present disclosure discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present disclosure provides a composition comprising one or more antibodies or antigen binding fragment thereof and one or more anti-oxidants such as methionine. The present disclosure further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the disclosure may be administered in various ranges. These include about 5 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the antibody or the antigen binding portion thereof of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or antigen-binding portion thereof of the present disclosure will comprise multiple doses of the selected drug product over a period of weeks or months. More specifically, the antibody or antigen-binding portion thereof of the present disclosure may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 mg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml, 900 μg/ml or 1 mg/ml. In other preferred embodiments, the concentrations of the antibody or the antigen binding portion thereof will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml

### Applications of the Disclosure

The antibodies, antibody compositions and methods of the present disclosure have numerous in vitro and in vivo utilities involving, for example, detection of P-cadherin or enhancement of immune response. For example, these molecules can be administered to cells in culture, in vitro or ex vivo, or to human subjects, e.g., in vivo, to enhance immunity in a variety of situations. The immune response can be modulated, for instance, augmented, stimulated or up-regulated.

For instance, the subjects include human patients in need of enhancement of an immune response. The methods are particularly suitable for treating human patients having a disorder that can be treated by augmenting an immune response (e.g., the T-cell mediated immune response). In a particular embodiment, the methods are particularly suitable for treatment of cancer in vivo. To achieve antigen- specific enhancement of immunity, the anti-P-cadherin antibodies can be administered together with an antigen of interest or the antigen may already be present in the subject to be treated (e.g., a tumor-bearing or virus-bearing subject). When antibodies to P-cadherin are administered together with another agent, the two can be administered in either order or simultaneously.

The present disclosure further provides methods for detecting the presence of human P-cadherin antigen in a sample, or measuring the amount of human P-cadherin antigen, comprising contacting the sample, and a control sample, with a human monoclonal antibody, or an antigen binding portion thereof, which specifically binds to human P-cadherin, under conditions that allow for formation of a complex between the antibody or portion thereof and human P-cadherin. The formation of a complex is then detected, wherein a difference complex formation between the sample compared to the control sample is indicative of the presence of human P-cadherin antigen in the sample. Moreover, the anti-P-cadherin antibodies of the disclosure can be used to purify human P-cadherin via immunoaffinity purification.

### Treatment of Disorders Including Cancers

In some aspects, the present disclosure provides a method of treating a disorder or a disease in a mammal, which comprises administering to the subject (for example, a human) in need of treatment a therapeutically effective amount of the antibody or antigen-binding portion thereof as disclosed herein. The disorder or disease may be a cancer.

A variety of cancers where P-cadherin is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In some embodiments, the cancer is a P-cadherin positive solid tumor. In some specific embodiments, the cancer is colorectal cancer. In some other embodiments, the cancer is breast cancer, prostate cancer or NSCLC.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliierative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/ Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

### Stimulation of an Immune Response

In some aspects, the disclosure also provides a method of enhancing (for example, stimulating) an immune response in a subject comprising administering an antibody or an antigen binding portion thereof of the disclosure to the subject such that an immune response in the subject is enhanced. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4⁺ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-2 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein.

The antibody or antigen-binding portion thereof may be used alone as a monotherapy, or may be used in combination with chemical therapies, radiotherapies, targeted therapies or cell immunotherapies etc.

### Combined Use with Chemotherapies

The antibody or antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the present disclosure. In other embodiments, the disclosed anti-cancer agents will be given in combination with site-specific conjugates comprising a different therapeutic agent as set forth above.

As used herein the term “cytotoxic agent” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific constructs of the present disclosure (either as a component of a site specific conjugate or in an unconjugated state) include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, OR), razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

### Combined Use with Radiotherapies

The present disclosure also provides for the combination of the antibody or antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed antibodies may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Diagnosis

The disclosure provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with an antibody as described herein and detecting presence or absence, or level of association, of the antibody to bound or free target molecules in the sample. In some embodiments, the antibody will comprise a detectable label or reporter molecule as described herein.

In some embodiments, the association of the antibody with particular cells in the sample can denote that the sample may contain tumorigenic cells, thereby indicating that the individual having cancer may be effectively treated with an antibody as described herein.

Samples can be analyzed by numerous assays, for example, radioimmunoassays, enzyme immunoassays (e.g. ELISA), competitive-binding assays, fluorescent immunoassays, immunoblot assays, Western Blot analysis and flow cytometry assays. Compatible in vivo theragnostic or diagnostic assays can comprise art recognized imaging or monitoring techniques, for example, magnetic resonance imaging, computerized tomography (e.g. CAT scan), positron tomography (e.g., PET scan), radiography, ultrasound, etc., as would be known by those skilled in the art.

### Pharmaceutical Packs and Kits

Pharmaceutical packs and kits comprising one or more containers, comprising one or more doses of the antibody or antigen-binding portion thereof are also provided. In certain embodiments, a unit dosage is provided wherein the unit dosage contains a predetermined amount of a composition comprising, for example, the antibody or antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed antibody is used for treating the neoplastic disease condition of choice.

The present disclosure also provides kits for producing single-dose or multi-dose administration units of antibodies and, optionally, one or more other anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed antibodies in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the antibody and, optionally, one or more anti-cancer agents in the same or different containers. The kits may also contain other pharmaceutically acceptable formulations, either for diagnosis or combined therapy. For example, in addition to the antibody or antigen-binding portion thereof of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the antibody or antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the antibodies and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

### Sequence Listing Summary

The CDRs, framework regions, constant region sequences of 7 antibodies as exemplified in the present disclosure are listed in the tables below.

TABLE A
Amino acid sequences of the VH region

| VH | FRW1 | CDR1 | FRW2 | CDR2 | FRW3 | CDR3 | FRW4 |
|---|---|---|---|---|---|---|---|
| W3195- | SEQ | SEQ | SEQ | SEQ | SEQ | SEQ ID | SEQ |
| 1.53.1- | ID | ID | ID | ID | ID | NO: 6 | ID |
| uIgG1L | NO: | NO: | NO: | NO: | NO: | DRRTGNS | NO: |
| (paren- | 1 | 2 | 3 | 4 | 5 | LPFDN | 7 |
| tal | QVQL | GGSV | WVRQ | YIYY | RVII | | WGQG |
| anti- | QESG | ISDN | PPGK | RGST | SIDT | | TLVT |
| body) | PGLV | YYWT | GLEF | NYNP | SRNQ | | VSS |
| | KPSE | | IG | SLKN | FSLD | | |
| | TLSL | | | | LNSV | | |
| | TCTV | | | | TAAD | | |
| | S | | | | TAVY | | |
| | | | | | YCAR | | |
| W3195- | SEQ | SEQ | SEQ | SEQ | SEQ | SEQ ID | SEQ |
| 1.53.1- | ID | ID | ID | ID | ID | NO: 6 | ID |
| p1- | NO: | NO: | NO: | NO: | NO: | | NO: |
| uIgG1L | 1 | 2 | 3 | 4 | 5 | | 7 |
| (p1) | | | | | | | |
| W3195- | SEQ | SEQ | SEQ | SEQ | SEQ | SEQ ID | SEQ |
| 1.53.1- | ID | ID | ID | ID | ID | NO: 6 | ID |
| p3- | NO: | NO: | NO: | NO: | NO: | | NO: |
| uIgG1L | 1 | 2 | 3 | 4 | 5 | | 7 |
| (p3) | | | | | | | |
| W3195- | SEQ | SEQ | SEQ | SEQ | SEQ | SEQ ID | SEQ |
| 1.53.1- | ID | ID | ID | ID | ID | NO: 8 | ID |
| p4- | NO: | NO: | NO: | NO: | NO: | DRRTGNA | NO: |
| uIgG1V320 | 1 | 2 | 3 | 4 | 5 | LPFDN | 7 |
| (p4) | | | | | | | |
| W3195- | SEQ | SEQ | SEQ | SEQ | SEQ | SEQ ID | SEQ |
| 1.53.1- | ID | ID | ID | ID | ID | NO: 9 | ID |
| p5- | NO: | NO: | NO: | NO: | NO: | DRRTGQS | NO: |
| uIgG1V320 | 1 | 2 | 3 | 4 | 5 | LPFDN | 7 |
| (p5) | | | | | | | |
| W3195- | SEQ | SEQ | SEQ | SEQ | SEQ | SEQ ID | SEQ |
| 1.53.1- | ID | ID | ID | ID | ID | NO: 10 | ID |
| p6- | NO: | NO: | NO: | NO: | NO: | DRRTGTS | NO:5 |
| uIgG1V320 | 1 | 2 | 3 | 4 | 5 | LPFDN | 7 |
| (p6) | | | | | | | |
| W3195- | SEQ | SEQ | SEQ | SEQ | SEQ | SEQ ID | SEQ |
| 1.53.1- | ID | ID | ID | ID | ID | NO: 11 | ID |
| p7- | NO: | NO: | NO: | NO: | NO: | DRRTGSS | NO: |
| uIgG1V320 | 1 | 2 | 3 | 4 | | LPFDN | 7 |
| (p7) | | | | | | | |

TABLE B
Amino acid sequences of the VL region

| VL | FRW1 | CDR1 | FRW2 | CDR2 | FRW3 | CDR3 | FRW4 |
|---|---|---|---|---|---|---|---|
| W3195- | SEQ ID | SEQ | SEQ | SEQ | SEQ | SEQ | SEQ |
| 1.53.1- | NO: 12 | ID | ID | ID | ID | ID | ID |
| uIgG1L | SYELTQPP | NO: | NO: | NO:15 | NO: | NO: | NO: |
| (paren- | SVSVSPG | 13 | 14 | QDNK | 16 | 17 | 18 |
| tal | QTANITC | SGDK | WYQQ | RPS | GFPE | QAWD | FGGG |
| anti- | | LGDK | KSGQ | | RFSG | SSIV | TKLT |
| body) | | FAS | SPVV | | SNSG | V | VL |
| | | | VIY | | NTAT | | |
| | | | | | LTIS | | |
| | | | | | GTQA | | |
| | | | | | MDEA | | |
| | | | | | DYYC | | |
| W3195- | SEQ ID | SEQ | SEQ | SEQ | SEQ | SEQ | SEQ |
| 1.53.1- | NO: 19 | ID | ID | ID | ID | ID | ID |
| p1- | SYELTQPP | NO: | NO: | NO: | NO: | NO: | NO: |
| uIgG1L | SVSVSPG | 13 | 14 | 15 | 16 | 17 | 18 |
| (p1) | QTAQITC | | | | | | |
| W3195- | SEQ ID | SEQ | SEQ | SEQ | SEQ | SEQ | SEQ |
| 1.53.1- | NO: 20 | ID | ID | ID | ID | ID | ID |
| p3- | SYELTQPP | NO: | NO: | NO: | NO: | NO: | NO: |
| uIgG1L | SVSVSPG | 13 | 14 | 15 | 16 | 17 | 18 |
| (p3) | QTANIAC | | | | | | |
| W3195- | same as | SEQ | SEQ | SEQ | SEQ | SEQ | SEQ |
| 1.53.1- | SEQ ID | ID | ID | ID | ID | ID | ID |
| p4- | NO: 19 | NO: | NO: | NO: | NO: | NO: | NO: |
| uIgG1V320 | | 13 | 14 | 15 | 16 | 17 | 18 |
| (p4) | | | | | | | |
| W3195- | same as | SEQ | SEQ | SEQ | SEQ | SEQ | SEQ |
| 1.53.1- | SEQ ID | ID | ID | ID | ID | ID | ID |
| p5- | NO: 19 | NO: | NO: | NO: | NO: | NO: | NO: |
| uIgG1V320 | | 13 | 14 | 15 | 16 | 17 | 18 |
| (p5) | | | | | | | |
| W3195- | same as | SEQ | SEQ | SEQ | SEQ | SEQ | SEQ |
| 1.53.1- | SEQ ID | ID | ID | ID | ID | ID | ID |
| p6- | NO: 19 | NO: | NO: | NO: | NO: | NO: | NO: |
| uIgG1V320 | | 13 | 14 | 15 | 16 | 17 | 18 |
| (p6) | | | | | | | |
| W3195- | same as | SEQ | SEQ | SEQ | SEQ | SEQ | SEQ |
| 1.53.1- | SEQ ID | ID | ID | ID | ID | ID | ID |
| p7- | NO: 19 | NO: | NO: | NO: | NO: | NO: | NO: |
| uIgG1V320 | | 13 | 14 | 15 | 16 | 17 | 18 |
| (p7) | | | | | | | |

TABLE C
Amino acid sequences of the Variable regions and Constant regions

| | | | Heavy chain | Light chain |
|---|---|---|---|---|
| | | | Constant | Constant |
| | VH | VL | region | region |
| W3195-1.53.1- | SEQ ID NO: 21 | SEQ ID NO: 26 | SEQ ID NO: 29 | SEQ ID NO: 30 |
| uIgG1L (parental | QVQLQESGPG | SYELTQPPSVS | ASTKGPSVFPLAPSSK | GQPKAAPSVTLFPPS |
| antibody) | LVKPSETLSLT | VSPGQTANITC | STSGGTAALGCLVKD | SEELQANKATLVCLI |
| | CTVSGGSVISD | SGDKLGDKFA | YFPEPVTVSWNSGAL | SDFYPGAVTVAWKA |
| | NYYWTWVRQ | SWYQQKSGQS | TSGVHTFPAVLQSSG | DSSPVKAGVETTTPS |
| | PPGKGLEFIGY | PVVVIYQDNK | LYSLSSVVTVPSSSLG | KQSNNKYAASSYLSL |
| | IYYRGSTNYNP | RPSGFPERFSG | TQTYICNVNHKPSNT | TPEQWKSHRSYSCQ |
| | SLKNRVIISIDT | SNSGNTATLTI | KVDKRVEPKSCDKT | VTHEGSTVEKTVAPT |
| | SRNQFSLDLNS | SGTQAMDEAD | HTCPPCPAPELLGGPS | ECS |
| | VTAADTAVYY | YYCQAWDSSI | VFLFPPKPKDTLMISR | |
| | CARDRRTGNS | VVFGGGTKLT | TPEVTCVVVDVSHED | |
| | LPFDNWGQGT | VL | PEVKFNWYVDGVEV | |
| | LVTVSS | | HNAKTKPREEQYNST | |
| | | | YRVVSVLTVLHQDW | |
| | | | LNGKEYKCKVSNKA | |
| | | | LPAPIEKTISKAKGQP | |
| | | | REPQVYTLPPSREEM | |
| | | | TKNQVSLTCLVKGFY | |
| | | | PSDIAVEWESNGQPE | |
| | | | NNYKTTPPVLDSDGS | |
| | | | FFLYSKLTVDKSRWQ | |
| | | | QGNVFSCSVMHEAL | |
| | | | HNHYTQKSLSLSPGK | |
| W3195-1.53.1- | SEQ ID NO: 21 | SEQ ID NO: 27 | | |
| p1-uIgG1L (p1) | | SYELTQPPSVS | | |
| | | VSPGQTAQITC | | |
| | | SGDKLGDKFA | | |
| | | SWYQQKSGQS | | |
| | | PVVVIYQDNK | | |
| | | RPSGFPERFSG | | |
| | | SNSGNTATLTI | | |
| | | SGTQAMDEAD | | |
| | | YYCQAWDSSI | | |
| | | VVFGGGTKLT | | |
| | | VL | | |
| W3195-1.53.1- | SEQ ID NO: 21 | SEQ ID NO: 28 | | |
| p3-uIgG1L (p3) | | SYELTQPPSVS | | |
| | | VSPGQTANIAC | | |
| | | SGDKLGDKFA | | |
| | | SWYQQKSGQS | | |
| | | PVVVIYQDNK | | |
| | | RPSGFPERFSG | | |
| | | SNSGNTATLTI | | |
| | | SGTQAMDEAD | | |
| | | YYCQAWDSSI | | |
| | | VVFGGGTKLT | | |
| | | VL | | |
| W3195-1.53.1- | SEQ ID NO: 22 | SEQ ID NO: 27 | SEQ ID NO: 31 | |
| p4-uIgG1V320 | QVQLQESGPG | | ASTKGPSVFPLAPSSK | |
| (p4) | LVKPSETLSLT | | STSGGTAALGCLVKD | |
| | CTVSGGSVISD | | YFPEPVTVSWNSGAL | |
| | NYYWTWVRQ | | TSGVHTFPAVLQSSG | |
| | PPGKGLEFIGY | | LYSLSSVVTVPSSSLG | |
| | IYYRGSTNYNP | | TQTYICNVNHKPSNT | |
| | SLKNRVIISIDT | | KVDKRVEPKSCDKT | |
| | SRNQFSLDLNS | | HTCPPCPAPEAAGGP | |
| | VTAADTAVYY | | SVFLFPPKPKDTLMIS | |
| | CARDRRTGNA | | RTPEVTCVVVDVSHE | |
| | LPFDNWGQGT | | DPEVKFNWYVDGVE | |
| | LVTVSS | | VHNAKTKPREEQYNS | |
| | | | TYRVVSVLTVLHQD | |
| | | | WLNGKEYKCKVSNK | |
| | | | ALPAPIEKTISKAKGQ | |
| | | | PREPQVYTLPPSREE | |
| | | | MTKNQVSLTCLVKG | |
| | | | FYPSDIAVEWESNGQ | |
| | | | PENNYKTTPPVLDSD | |
| | | | GSFFLYSKLTVDKSR | |
| | | | WQQGNVFSCSVMHE | |
| | | | ALHNHYTQKSLSLSP | |
| | | | GK | |
| W3195-1.53.1- | SEQ ID NO: 23 | SEQ ID NO: 27 | | |
| p5-uIgG1V320 | QVQLQESGPG | | | |
| (p5) | LVKPSETLSLT | | | |
| | CTVSGGSVISD | | | |
| | NYYWTWVRQ | | | |
| | PPGKGLEFIGY | | | |
| | IYYRGSTNYNP | | | |
| | SLKNRVIISIDT | | | |
| | SRNQFSLDLNS | | | |
| | VTAADTAVYY | | | |
| | CARDRRTGQS | | | |
| | LPFDNWGQGT | | | |
| | LVTVSS | | | |
| W3195-1.53.1- | SEQ ID NO: 24 | SEQ ID NO: 27 | | |
| p6-uIgG1V320 | QVQLQESGPG | | | |
| (p6) | LVKPSETLSLT | | | |
| | CTVSGGSVISD | | | |
| | NYYWTWVRQ | | | |
| | PPGKGLEFIGY | | | |
| | IYYRGSTNYNP | | | |
| | SLKNRVIISIDT | | | |
| | SRNQFSLDLNS | | | |
| | VTAADTAVYY | | | |
| | CARDRRTGTS | | | |
| | LPFDNWGQGT | | | |
| | LVTVSS | | | |
| W3195-1.53.1- | SEQ ID NO: 25 | SEQ ID NO: 27 | | |
| p7-uIgG1V320 | QVQLQESGPG | | | |
| (p7) | LVKPSETLSLT | | | |
| | CTVSGGSVISD | | | |
| | NYYWTWVRQ | | | |
| | PPGKGLEFIGY | | | |
| | IYYRGSTNYNP | | | |
| | SLKNRVIISIDT | | | |
| | SRNQFSLDLNS | | | |
| | VTAADTAVYY | | | |
| | CARDRRTGSS | | | |
| | LPFDNWGQGT | | | |
| | LVTVSS | | | |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, Antigens, Benchmark Antibodies and Cell Lines

### 1.1 Preparation of Materials

Information on the commercially available materials used in the examples is provided in Table 1A.

TABLE 1A

| Materials | Vendor | Cat. |
|---|---|---|
| NCI-H1650 | ATCC | CRL-5883 |
| DU-145 | ATCC | HTB-81 |
| HCC-1954 | ATCC | CRL-2338 |
| HCT-116 | ATCC | CCL-247 |
| Expi293F ™ Cells | Thermo Fisher | A14527 |
| ExpiFectamine293 transfection kit | Thermo Fisher | A14524 |
| Expi293F ™ expression medium | Thermo Fisher | A1435101 |
| Lipofectamine ™ 2000 Transfection | Thermo Fisher | 11668019 |
| Reagent | | |
| FreeStyle ™ 293-F Cells | Thermo Fisher | R79007 |
| FreeStyle ™ 293 Expression Medium | Thermo Fisher | 12338002 |
| FreeStyle ™ CHO Expression Medium | GIBCO | 12651014 |
| Fetal bovine serum (FBS) | Corning | 35-076-CV |
| Opti-MEM | Thermo Fisher | 31985070 |
| Ni column | GE healthcare | 173712 |
| HPLC-SEC | TOSOH | 0008541 |
| NuPAGE4%-12% Bis-Tris Gel | Thermo Fisher | NP0322BOX |
| Recombinant Human P-cadherin Fc | R&D systems | 861-PC-100 |
| Chimera Protein(His) | | |
| Recombinant Human Cadherin-3/CDH3 | Novoprotein | C968 |
| (C-6His) | Scientific | |
| Recombinant Mouse Cadherin-3/P- | Novoprotein | CK97 |
| cadherin/CDH3 (C-Fc) | Scientific | |
| Human/Mouse E-Cadherin Antibody | R&D | AF648 |
| (WBP319-cAb2 (R&D)) | | |
| CD325 (N-Cadherin) Monoclonal | eBioscience | 14-3259-82 |
| Antibody (8C11) (WBP319-cAb4) | | |

### 1.2 Construction of Expression Vector of Soluble Antigens

The amino acid sequences encoding the extracellular domain of human P-cadherin (Uniprot ID: P22223, aa 108-654), domain 1 (aa 108-236), domain 1 & 2 (aa 108-348), domain 1&2&3 (108-461) and domain 1&2&3&4 (108-550) respectively were first codon optimized for mammalian expression and then synthesized by GENEWIZ (SuZhou, CHINA). The DNA segment was then sub-cloned into the pcDNA3.3 expression vector with 6xHis at the C-terminal. Protein samples of human, cyno and mouse P-cadherin were also purchased from Sino Biological.

TABLE 1B
Coding of the antigens

| Antigen Code | Antigen |
|---|---|
| hPro1 | Human P-cadherin |
| W319-hPro1.D1.ECD.hFc | Human P-cadherin ECD domain 1 |
| W319-hPro1.D12.ECD.hFc | Human P-cadherin ECD domain |
| (WT) | 1&2 |
| W319-hPro1.D123.ECD.hFc | Human P-cadherin ECD domain |
| (WT) | 1&2&3 |
| W319-hPro1.D1234.ECD.hFc | Human P-cadherin ECD domain |
| (WT) | 1&2&3&4 |
| W319-hPro1.ECD.hFc (WT)-P3 | Human P-cadherin ECD |
| cynoPro1 | Cyno P-cadherin |
| WBP319-hPro2.ECD.His | Human E-Cadherin |
| WBP319-hPro3.ECD.hFcHis | Human N-Cadherin |

### 1.3 Construction of Expression Vector of BMK Antibodies

The amino acid sequences encoding the variable domains of anti-P-cadherin antibodies used as benchmark antibodies (WBP319-BMK1, WBP319-BMK2 and WBP319-BMK4) in the following experiments were first codon optimized for mammalian expression and then synthesized by GENEWIZ (SuZhou, CHINA). The DA segments were then sub-cloned into pcDNA3.4 expression vectors with constant region of human IgG1.

TABLE 2
Benchmark antibody information

| Antibody | | | Sequence | Molecular |
|---|---|---|---|---|
| Code | Company | Patent NO. | ID | Name |
| WBP319- | Pfizer | WO2006114704A2 | Seq 71, | PF-03732010 |
| BMK1 | | | 84 | |
| WBP319- | Pfizer | US20160002357A1 | Seq 90, | PF-06671008 |
| BMK2 | | | 91 | |
| WBP319- | Norvatis | WO2016075670A1 | Seq ID 8, | PCA062 |
| BMK4 | | | 18 | |

### 1.4 Small Scale Expression of Protein

The plasmids containing VH and VL gene were co-transfected into Expi293 cells (Thermofisher, A14635). The cells were cultured for 5 days following the manufacturer suggested protocol. The supernatants were collected and analyzed by SDS-PAGE.

The plasmids containing VH and VL gene were co-transfected into ExpiCHO cells (Thermofisher, A29133). The cells were cultured for 10 days following the manufacturer suggested protocol. The supernatants were collected and analyzed by SDS-PAGE.

### 1.5 Purification of Fc-Tagged Protein

The supernatant of Expi293 cells or ExpiCHO cells-expressing target proteins was collected and filtered for purification using either Protein A column (GE Healthcare, Cat. 175438) or Protein G column (GE Healthcare, Cat. 170618). The concentration of purified Fc-tagged proteins was determined by absorbance at 280 nm. The size and purity were tested by SDS-PAGE and SEC-HPLC, respectively; and then stored at −80° C.

### 1.5 Cell Pool/Line Generation

### Generation of Target-Expressing Cell Pool

Human P-cadherin-expressing cell pools were generated. Briefly, CHO-K1 were transfected with pcDNA3.3 expression vector containing full-length of P-cadherin using Lipofectamine 2000 transfection kit (ThermoFisher-11668027) according to manufacturer's protocol. At 48-72 hours post transfection, the cells were subcultured to T125 flask in selective media. After two or three passages of selection, stable cell pool were obtained and expression level was determined by FACS using anti-P-cadherin antibody.

### Generation of Target-Expressing Cell Lines

Lipofectamine 2000 was used to transfect CHO-K1 cells with the expression vector containing gene encoding full length human P-cadherin. The transfected cells were cultured in medium containing proper selection pressure. Human P-cadherin high expression stable cell line (W319-CHOK1.hPro1.FL.S114) were obtained by limiting dilution.

### Example 2

### Antibody Hybridoma Generation, Screening and Optimization

### 2.1 Immunization

OmniRat is a transgenic rat developed by Open Monoclonal Technology Company, which carries a chimeric human/rat IgH locus. 4 OMT rats at age of 6-8 weeks were immunized with 40-60 μg human P-Cadherin ECD protein and 200-600 μg plasmid DNA antigen/animal. The adjuvant mixture includes Adju-Phos, CpG-ODN and Titer-Max. The animals were injected once every other week via footpad, subcutaneous, intra-peritoneal, intramuscular, intradermal routes, in a total of 9 injections. The serum titer was measured by ELISA and FACS. When the serum titer was sufficiently high (≥1:24,300), the animal with the highest titer were given a final boost with protein in sterilized PBS without adjuvant. After 3 days (72 hours), the animals were euthanized, lymph nodes and spleen were extracted and used for cell fusion.

### 2.2 Serum Titer Detection

Cell-based ELISA assay was used to measure serum antibody titers against given antigen. Briefly, P-cadherin transfected CHO-K1 cell line (W319-CHOK1.hPro1.FL.S114) were seeded in 96-well plates (CORNING) at the density of 5×10⁴ cells/well, kept in a 37° C. 5% CO₂ incubator overnight, and then blocked with blocking buffer (1×PBS (Ca+/Mg+)/5% milk) for 1 h at room temperature. The plates were then washed and incubated with hybridoma supernatant for 1 h at room temperature. The plates were then washed and subsequently incubated with secondary antibody goat anti-rat IgG-Fc-HRP (Bethyl) for 1 h. After washing, TMB substrate was added and the interaction was stopped by 2M HCl. The absorbance at 450 nm was read using a microplate reader (Molecular Device). Serum titer was determined at 3-folds background.

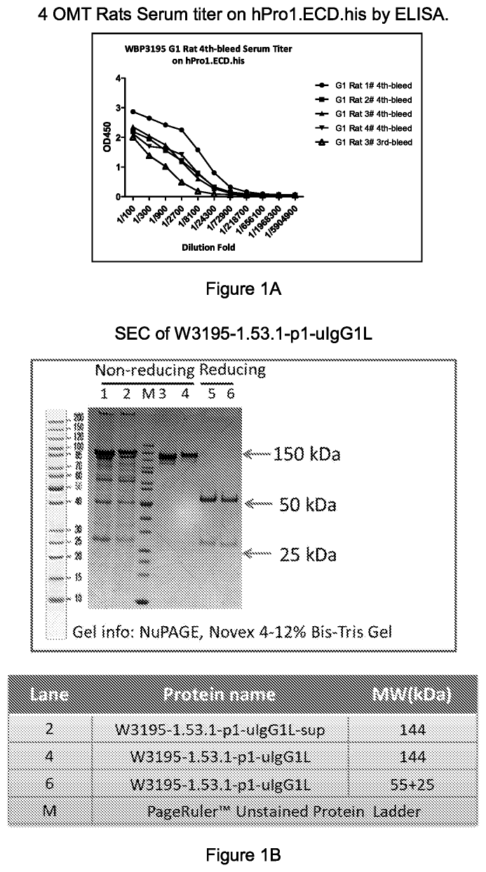

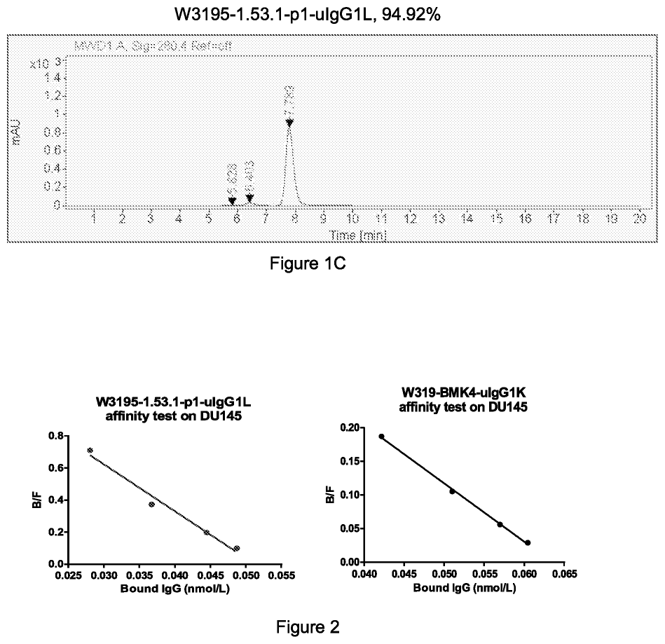

4 OMT rats showed strong immune response with the serum titer of 1:72900-1:218700 against hPro1. The serum titer data was shown in FIG. 1A.

### 2.3 Hybridoma Generation

Lymph nodes from immunized animal were homogenized and filtered to remove blood clots and cell debris. Sp2/0 myeloma cells in logarithmic growth were collected and centrifuged. B cells and Sp2/0 myeloma cells were treated separately with pronase solution and the reaction was stopped by FBS. The cells were washed and counted. B cells were fused with Sp2/0 myeloma cells at 1:1 ratio in electric fusion solution following general electro-fusion procedures. The fused cells were resuspended in DMEM medium supplemented with 20% FBS and 1×HAT, and then transferred into 96-well plates (CORNING). The fused cells were kept in a 37° C. 5% CO₂ incubator for 10˜14 days.

### 2.4 Antibody Screening and Subcloning

Cell-based ELISA assay was used as first screen method to test the binding of hybridoma supernatants to hPro1 antigen; flow cytometry analysis was performed on P-cadherin transfected CHO-K1 cell line (W319-CHOK1.hPro1.FL.S114), parental CHO-K1 cell line, tumor cells that express antigen and tumor cells that don't express antigen, in order to confirm the binding of hybridoma supernatants to hPro1 antigen and counter screening; and traditional ELISA assay was used to test the binding of hybridoma supernatants to mouse P-cadherin and cyno P-cadherin antigen.

After a serial comprehensive screening process, one positive hybridoma was identified and subcloned. The hybridoma cells in logarithmic growth were counted and 200 cells were added to 1.5 ml semi-solid-HAT media. The cells were mixed gently in vortex oscillators for 5-10 seconds and then seeded in 6-well plates (CORNING). The plates were kept in a 37° C. 5% CO₂ incubator for 7-8 days. Each visible single colony was picked into 96-well plates (CORNING) with DMEM medium supplemented with 10% FBS. After 2˜3 days, the cell supernatant were collected and screened. After subcloning, dozens of single clones were obtained.

The total RNA of the hybridoma cell sample is first extracted, following the instruction of the TaKaRa MiniBEST Universal RNA Extraction Kit. The SMART RACE cDNA Amplication Kit from Clonetech is then used to convert RNA to cDNA. The VH and VL domain DNA sequences were then amplified from cDNA with 30 cycles of PCR, each with denaturation at 94° C. for 30 sec, anneal at 60 C for 30 sec, then elongation at 72° C. for 30 sec. The PCR product is then sub-cloned to TA-cloning vector, then sent for GENEWIZ (SuZhou, CHINA) for sequencing. Once sequencing data confirms monoclonality of the hybridoma cell sample, the amino acid sequences of the VH and VL domains were codon optimized for mammalian expression then synthesized by GENEWIZ (SuZhou, CHINA). The DNA segments were then sub-cloned into pcDNA3.4 expression vectors with constant region of human IgG1.

### 2.5 Antibody Optimization

### 2.5.1 IgG Conversion

Once sequencing data confirms monoclonality of the hybridoma cell sample, the amino acid sequences of the VH and VL domains were codon optimized for mammalian expression then synthesized by GENEWIZ (SuZhou, CHINA). The DNA segments were then sub-cloned into pcDNA3.4 expression vectors with constant region of human IgG1. The obtained antibody was named as W3195-1.53.1-uIgG1L and used as the parental antibody for further optimization.

### 2.5.2 PTM Removal

Generally, PTM modification mainly include isomerization, deamination, glycosylation and oxidation in antibody discovery, all of them have the typical site, such as DG for isomerization, NG for deamination, N*T/S (* stand for other amino acid except P or D) for glycosylation and M or C for oxidation and so on. Once the PTM sites are found in our antibody sequence, especially in the key region like CDR3, PTM removal are needed to avoid the potential risk of PTM modification.

The following PTM-removal antibodies were obtained, namely, W3195-1.53.1-p1-uIgG1L, W3195-1.53.1-p3-uIgG1L, W3195-1.53.1-p4-uIgG1L, W3195-1.53.1-p5-uIgG1L, W3195-1.53.1-p6-uIgG1L and W3195-1.53.1-p7-uIgG1L. N19Q (VL domain) mutation was chosen to remove the PTM site of W3195-1.53.1-p1-uIgG1L.

Further, L234A/L235A substitution was introduced into the IgG1 constant region of W3195-1.53.1-p1-uIgG1L, W3195-1.53.1-p3-uIgG1L, W3195-1.53.1-p4-uIgG1L, W3195-1.53.1-p5-uIgG1L, W3195-1.53.1-p6-uIgG1L and W3195-1.53.1-p7-uIgG1L, resulting in W3195-1.53.1-p1-uIgG1LV320, W3195-1.53.1-p3-uIgG1LV320, W3195-1.53.1-p4-uIgG1LV320, W3195-1.53.1-p5-uIgG1LV320, W3195-1.53.1-p6-uIgG1LV320 and W3195-1.53.1-p7-uIgG1LV320.

After PTM removal, FACS binding assay on hPro1 expressing DU-145 was performed to determine final lead. W3195-1.53.1-p1-uIgG1L mAb showed good binding to hPro1-expressing DU-145 cells with an EC50 of 0.07 nM, which was comparable with parental mAb before PTM removal.

### Example 3

### In Vitro Characterization of the Antibodies

### 3.1 SDS-PAGE and Size Exclusion Chromatography (SEC-HPLC)

Nu PAGE Bis-Tris Mini Gels 4-12% (Invitrogen), Nu PAGE MES SDS Running Buffer (20×) (Invitrogen), and the Simply Blue Safe Stain (Invitrogen) were used. The samples were mixed with the loading buffer, heated at 75° C. for 10 minutes, loaded on the gel and run at a constant voltage (200V) for 35 minutes. The gel was rinsed with water for 10 minutes, repeated for 3 times, then stained with staining buffer for 1 hour, destained with water for 1 hour and repeated for 3 times.

The purity of antibodies was tested by SEC-HPLC assay using Agilent 1260 Infinity HPLC. Briefly, 50 μL of antibody solution was injected on a TSKgel SuperSW3000 column using 50 mM sodium phosphate, 0.15 M NaCl, pH 7.0 as running buffer. The run time was 20 minutes. Peak retention times on the column were monitored at 280 nm. Data was analyzed using ChemStation software (V2.99.2.0).

Non-reducing and reducing bands were visible on the gel (FIG. 1B). The purity of the sample was 94.92% (FIG. 1C). The retention time around at 7.8 minutes indicating the protein monomer.

TABLE 3

| | Purity by | Yield | Molecular | |
|---|---|---|---|---|
| Antibody Name | SEC-HPLC | (mg/L) | Weight | PI |
| W3195-1.53.1-p1-uIgG1L | 94.92% | 265.62 | 144 | 8.10 |

### 3.2 Affinity to P-Cadherin (FACS)

The DU-145 (HTB-81) cells were seeded in 96-well U-bottom plates (BD) at a density of 5×10⁴ cells/well. Antibodies to be tested were serially diluted in 1×PBS/1% BSA and incubated with cells at 4° C. for 1 h. The plates were centrifuged and the supernatant was discarded. The cells were then incubated with Alexa647 conjugated goat anti-human IgG Fc (Jackson) at 4° C. in the dark for 30 min. After washing the cells were re-suspended in 100 μL 1×PBS/1% BSA, and fluorescence intensity was measured by flow cytometry (BD Canto II) and analyzed by FlowJo. The fluorescence intensity was converted to bound molecules/cell based on the quantitative beads standard curve (Quantum™ MESF Kits, Bangs Laboratories). K_{D} was calculated by Graphpad Prism5.

The result is shown in the Table 4 and FIG. 2. W3195-p1 antibody showed good affinity effect on hPro1 expressing DU-145 cells with a KD of 3.4E-11 M, which is better than W319-BMK4-uIgG1K (1.2E-10 M).

TABLE 4

| mAb | W3195-1.53.1-p1-uIgG1L | W319-BMK4-uIgG1K |
|---|---|---|
| Bmax (M) | 5.1E−11 | 6.4E−11 |
| KD (M) | 3.4E−11 | 1.2E−10 |
| r² | 0.98 | 1.00 |

### 3.3 Target Binding (FACS)

Binding of the test antibodies to P-cadherin expressed on cells was determined by flow cytometry analysis. In brief, NCI-H1650 (ATCC, #CRL-5883), HCT-116 (ATCC, #CCL-247) and DU-145 (ATCC, #HTB-81) cells were harvested with Versene (Invitrogen, #15040066) and diluted to 1×10⁶ cell s/ml in 1% BSA (Bovogen, # BSAS)/1×PBS (Ca+/Mg+) (Gibco, #14040-117). 1×10⁵ cells/well (100 μL) were added to each well of a 96-well U-plate (Corning, #3799) and centrifuged at 1500 rpm (Eppendorf, #5810R) for 5 minutes before removing the supernatant. Antibodies serially diluted in 1% BSA/1×PBS (Ca+/Mg+) were added at 100 μL/well to the pelleted cells and incubated at 4° C. for 1 hour. Anon-related hIgG1 antibody was used as an isotype control. Cells were washed two times with 180 μL/well of 1% BSA/1×PBS (Ca+/Mg+) by centrifugation at 1500 rpm for 5 minutes at 4° C. Pelleted cells were resuspended in 100 μL/well Alexa647 conjugated Goat anti-human IgG Fc (Jackson, #109-605-098) 1:500 diluted in 1% BSA/1×PBS (Ca+/Mg+) for 30 minutes at 4° C. in the dark. Cells were then washed two times as described above. After the final wash, cells were resuspended in 100 μL 1% BSA/1×PBS (Ca+/Mg+) and fluorescence values were measured with a FACS Canto II cytometer (BD Biosciences). The amount of cell surface bound anti-P-cadherin antibody was assessed by measuring the mean fluorescence (MFI). The FACS raw data were analyzed by FlowJo software, wells containing no antibody or secondary antibody only were used to establish background fluorescence. Binding EC50 values were obtained by the four-parameter non-linear regression analysis using GraphPad Prism6 software.

TABLE 5A

DU-145

| mAbs | EC50 (nM) | Max MFI |
|---|---|---|
| W3195-1.53.1-uIgG1L | 0.05 | 2092 |
| W3195-1.53.1-p1-uIgG1L | 0.07 | 2101 |
| W3195-1.53.1-p3-uIgG1L | 0.05 | 2136 |
| W319-BMK4.uIgG1K | 0.16 | 2715 |
| Isotype control | >100 | 263 |

TABLE 5B

NCI-H1650

| mAbs | EC50 (nM) | Max MFI |
|---|---|---|
| W3195-1.53.1-p1-uIgG1L | 0.22 | 5767 |
| W319-BMK4.uIgG1K | 0.19 | 7300 |
| Isotype control | >100 | 65.1 |

TABLE 5C

HCT-116

| mAbs | EC50 (nM) | Max MFI |
|---|---|---|
| W3195-1.53.1-p1-uIgG1L | 0.21 | 6121 |
| W319-BMK4.uIgG1K | 0.33 | 7651 |
| Isotype control | >100 | 402 |

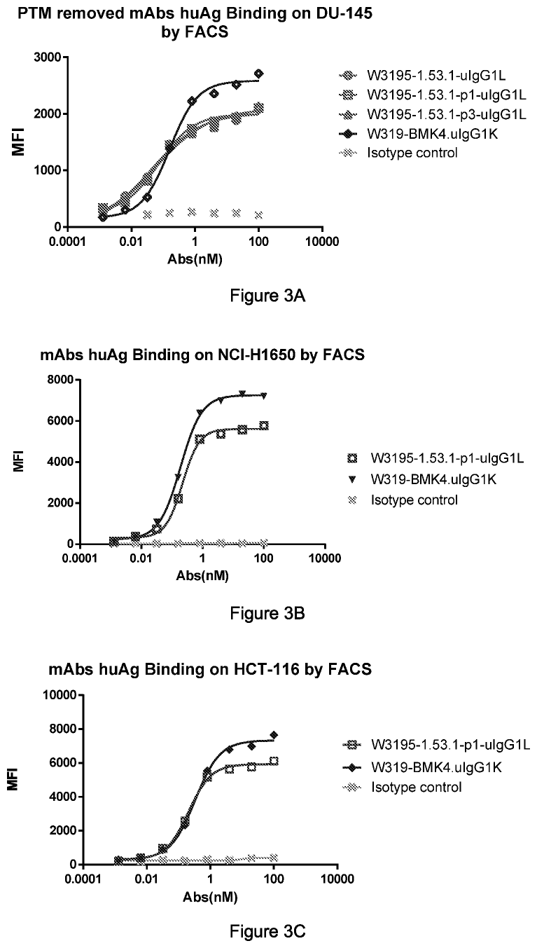

As shown in Tables 5A-C and FIGS. 3A-C, W3195-1.53.1-p1-uIgG1L mAb showed good binding to hPro1-expressing DU-145 cells with an EC50 of 0.07 nM, which was comparable with parental mAb before PTM removal and better than W319-BMK4.uIgG1K; showed binding to hPro1-expressing NCI-H1650 cells with an EC50 of 0.22 nM, comparable with W319-BMK4.uIgG1K (0.19 nM); and showed binding to hPro1-expressing HCT-116 cells with an EC50 of 0.21 nM, better than W319-BMK4.uIgG1K (0.33 nM). W3195-1.53.1-p3-uIgG1L mAb showed good binding to hPro1-expressing DU-145 cells with an EC50 of 0.05 nM, which was comparable with parental mAb before PTM removal.

### 3.4 Cross Species Binding (FACS)

Binding of the test antibodies to cynomolgus P-cadherin expressed on cells was determined by flow cytometry analysis. The procedure was the same as described above, except cynoP-cadherin over-expressing CHOK1 cells (transiently transfected, W319-CHOK1.cynoPro1.FL) were used.

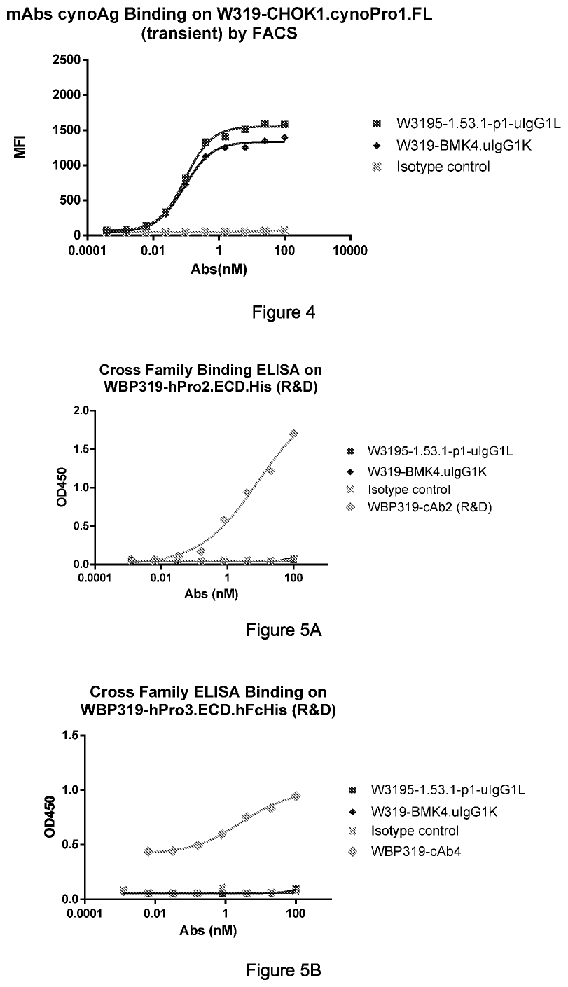

As indicated in Table 6 and FIG. 4, W3195-1.53.1-p1-uIgG1L mAb showed binding to cynoPro1 transient expressing W319-CHOK1.cynoPro1.FL cells with an EC50 of 0.095 nM, which was comparable with W319-BMK4.uIgG1K (0.088 nM).

TABLE 6

W391-CHOK1.cynoPro1.FL
(transient)

| mAbs | EC50 (nM) | Max MFI |
|---|---|---|
| W3195-1.53.1-p1-uIgG1L | 0.095 | 1582 |
| W319-BMK4.uIgG1K | 0.088 | 1396 |
| Isotype control | >100 | 76.3 |

### 3.5 Paralog/Specificity Binding (ELISA)

The binding of the antibodies to human E-Cadherin.ECD.His (R&D-8505-NC-050, WBP319-hPro2.ECD.His (R&D)) and human N-Cadherin.ECD.His (R&D-1388-NC-050, WBP319-hPro3.ECD.hFcHis (R&D)) were tested by a captured protein binding ELISA. 96-well high protein binding ELISA plates (Nunc MaxiSorp, ThermoFisher, Cat #: 442404) were coated overnight at 4° C. with 1 μg/mL capture antibody (THETM His Tag mAb, GenScript, Cat #: A00186) in Carbonate-bicarbonate buffer (20 mM Na2CO3, 180 mM NaHCO3, pH=9.2). All wells were washed three time with 300 μL per well of PBS/0.5‰ Tween-20 (v/v) and all the following wash steps in the assay were performed the same. The wells were then blocked for one hour with 3% milk (Shanghai Dingguo-DH220)/1×PBS (Ca+/Mg+) (Gibco, #14040-117) and washed three times, followed by binding of 1 μg/mL antigens in 3% milk/1×PBS (Ca+/Mg+) for 1 hour at room temperature and three washes afterwards. For the primary antibody binding, the testing antibodies including the BMKs and our lead antibodies serially diluted in 3% milk/1×PBS (Ca+/Mg+) were added to the relevant wells and incubated at room temperature for two hours. WBP319-cAb2 (R&D) and WBP319-cAb4 were included as positive control for the binding to E-Cadherin and N-Cadherin separately. Plates were washed three times prior to the addition of 100 μL of secondary antibody Goat-anti-human IgG Fc-HRP (Bethyl, Cat #: A80-304P, 5000-fold diluted), donkey anti-goat IgG (H&L)—HRP (Bethyl, Cat #: A90-231P, 10000-fold diluted) and Goat anti-Rat IgG cross-absorbed Fc-HRP (Bethyl, Cat #: A110-236P, 5000-fold diluted) in 3% milk/1×PBS (Ca+/Mg+). Plates were incubated at room temperature for one hour, followed by six washes as describe above.

For the binding detection, 100 μL Tetramethylbenzidine (TMB) Substrate solution (Invitrogen, Cat #: 002023) was added to all wells for 10 minutes before stopping the reaction with 100 μL 2M HCl. The extent of the testing Abs binding to WBP319-hPro2.ECD.His (R&D)) and WBP319-hPro3.ECD.hFcHis (R&D) were determined by measuring the OD450 absorbance using the SpectraMax® M5e microplate reader. Wherever appropriate, binding EC50 values were obtained by the four-parameter non-linear regression analysis using GraphPad Prism 5 software.

As shown in Table 7 and FIGS. 5A-B, W3195-1.53.1-p1-uIgG1L had no non-specific binding on WBP319-hPro2 & WBP319-hPro3 with EC50ₛ>100 nM.

TABLE 7

| | WBP319-hPro2.ECD.His (R&D) | | WBP319-hPro3.ECD.hfcHis (R&D) | |
|---|---|---|---|---|
| mAb | EC50 (nM) | Max OD450 | EC50 (nM) | Max OD450 |
| W3195-1.53.1-p1-uIgG1L | >100 | 0.06 | >100 | 0.10 |
| W319-BMK4.uIgG1K | >100 | 0.08 | >100 | 0.09 |
| Isotype control | >100 | 0.08 | >100 | 0.08 |
| WBP319-cAb2 (R&D) | 8.13 | 1.70 | — | — |
| WBP319-cAb4 | — | — | 3.24 | 0.95 |

### 3.6 Domain Determination Binding (ELISA)

The binding of the antibodies to W319-hPro1.D1.ECD.hFc, W319-hPro1.D12.ECD.hFc (WT), W319-hPro1.D123.ECD.hFc (WT), W319-hPro1.D1234.ECD.hFc (WT) (2.5) and W319-hPro1.ECD.hFc (WT)-P3 were tested by a direct protein binding ELISA. 96-well high protein binding ELISA plates (Nunc MaxiSorp, ThermoFisher, Cat #: 442404) were coated overnight at 4° C. with 1 μg/mL antigens in Carbonate-bicarbonate buffer (20 mM Na2CO3, 180 mM NaHCO3, pH=9.2). All wells were washed three time with 300 μL per well of PBS/0.5‰ Tween-20 (v/v) and all the following wash steps in the assay were performed the same. The wells were then blocked for one hour with 2% BSA (Bovogen, Cat #: BSAS)/1×PBS (Ca+/Mg+) (Gibco, #14040-117) and washed three times. For the primary antibody binding, the testing antibodies including the BMKs and our antibodies serially diluted in 2% BSA/1×PBS (Ca+/Mg+) were added to the relevant wells and incubated at room temperature for two hours. Plates were washed three times prior to the addition of 100 μL of secondary antibody Goat anti-Human-IgG-F(ab′)2-HRP (Jackson, Cat #: 109-035-097) 5000-fold diluted in 2% BSA/1×PBS (Ca+/Mg+). Plates were incubated at room temperature for one hour, followed by six washes as describe above.

For the binding detection, 100 μL Tetramethylbenzidine (TMB) Substrate solution (Invitrogen, Cat #: 002023) was added to all wells for 10 minutes before stopping the reaction with 100 μL 2M HCl. The extent of the testing Abs binding to W319-hPro1.D1.ECD.hFc, W319-hPro1.D12.ECD.hFc (WT), W319-hPro1.D123.ECD.hFc (WT), W319-hPro1.D1234.ECD.hFc (WT) and W319-hPro1.ECD.hFc (WT)-P3 were determined by measuring the OD450 absorbance using the SpectraMax® M5e microplate reader. Wherever appropriate, binding EC50 values were obtained by the four-parameter non-linear regression analysis using GraphPad Prism 5 software.

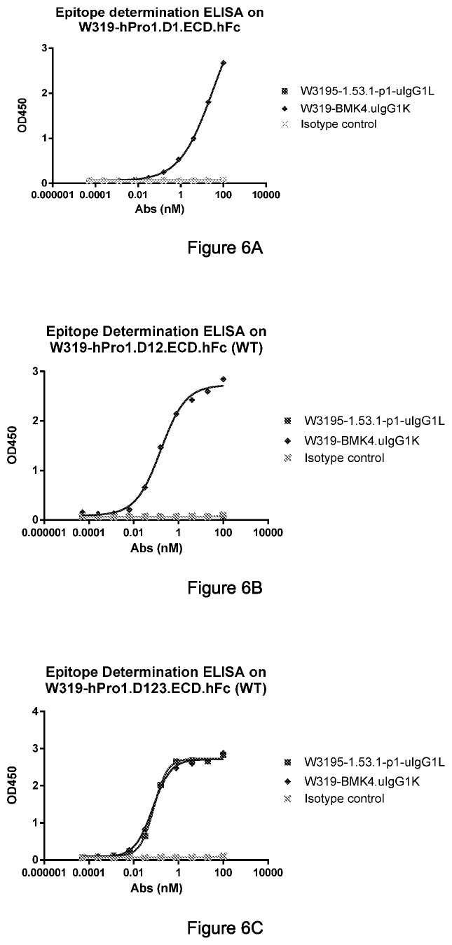

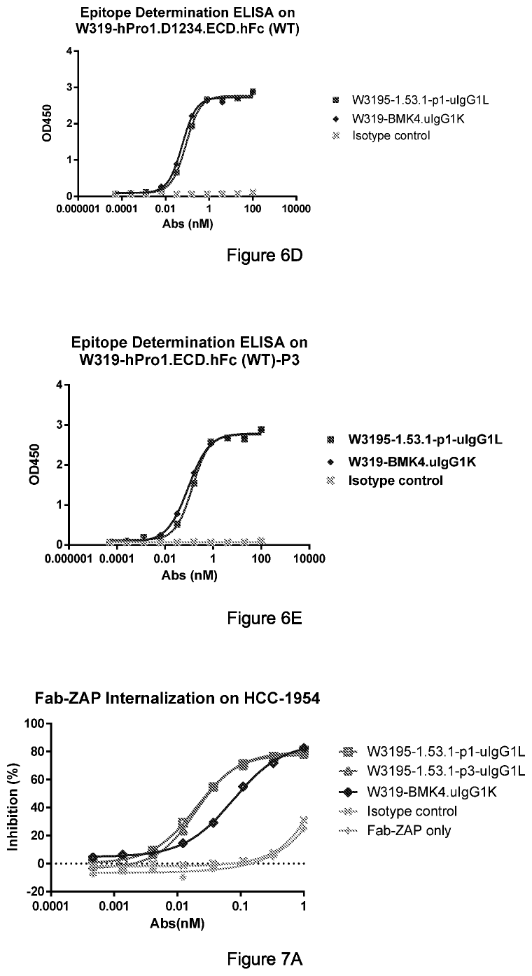

As indicated in Table 8 and FIGS. 6A-E, W3195-1.53.1-p1-uIgG1L showed binding on Domain 3 with an EC50 of 0.080 nM. Its binding epitope is different from BMKs.

TABLE 8

| | | | W319-hPro1.D12.ECD.hFc | | W319-hPro1.D123.ECD.hFc | |
|---|---|---|---|---|---|---|
| | W319-hPro1.D1.ECD.hFc | | (WT) | | (WT) | |
| mAb | EC50 (nM) | Max OD450 | EC50 (nM) | Max OD450 | EC50 (nM) | Max OD450 |
| W3195-1.53.1-p1-uIgG1L | >100 | 0.07 | >100 | 0.08 | 0.080 | 2.83 |
| W319-BMK4.uIgG1K | 32 | 2.68 | 0.16 | 2.84 | 0.071 | 2.88 |
| Isotype control | >100 | 0.08 | >100 | 0.10 | >100 | 0.11 |

| | | | |
|---|---|---|---|
| nc | 0.12 | 0.10 | 0.11 |
| Blank | 0.07 | 0.06 | 0.06 |

| | W319-hPro1.D1234.ECD.hFc | | W319-hPro1.ECD.hFc | |
|---|---|---|---|---|
| | (WT) | | (WT)-P3 | |
| mAb | EC50 (nM) | Max OD450 | EC50 (nM) | Max OD450 |
| W3195-1.53.1-p1-uIgG1L | 0.087 | 2.88 | 0.14 | 2.88 |
| W319-BMK4.uIgG1K | 0.058 | 2.87 | 0.092 | 2.89 |
| Isotype control | >100 | 0.10 | >100 | 0.10 |

| | | |
|---|---|---|
| nc | 0.12 | 0.12 |
| Blank | 0.06 | 0.06 |

### 3.7 Antibody Mediated Internalization Assay (Fab-ZAP)

Fab-ZAP is a chemical conjugate of goat anti-human monovalent antibody and the ribosome-inactivating protein saporin. The Fab-ZAP antibodies used to measure human antibody internalization were affinity-purified polyclonal antibodies against both the heavy and light chain of human IgG. Fab-ZAP is used for screening human IgG antibodies for internalization. One day before the assay day, HCC-1954 (ATCC, CRL-2338) cells were plated at 4000 cells per well into 96-well clear bottom black plates (Greinier, #655090) in 50 μL RPMI1640 complete medium (Gibco, #22400-089) containing 10% FBS (Hyclone, #SH30084.03). On Day 1, the purified antibodies and Fab-Zap (Advanced Targeting Systems, IT-51) were mixed at a ratio of Fab-Zap: Ab=3:1 (unit: mol/L) and incubated for 30 minutes at 37° C. The Ab-Fab-Zap complex were then serially diluted with assay medium and added onto the HCC-1954 cells in 96 well plate. The cells were kept in a 37° C., 5% CO2 incubator for another 4 days before assessing cell viability using Cell Titer Glo (Promega, #G7573). 50 μL of Cell Titer Glo solution was added to each well and incubated at room temperature with gentle shaking for 10 minutes. The amount of luminescence was determined using Envision (PerkinElmer). The cytotoxicity effect obtained with each antibody was calculated by comparing the luminescence values obtained with Cell only control wells. Four-parameter non-linear regression analysis was used to obtain IC50 values for antibody internalization using GraphPad Prism6 software.

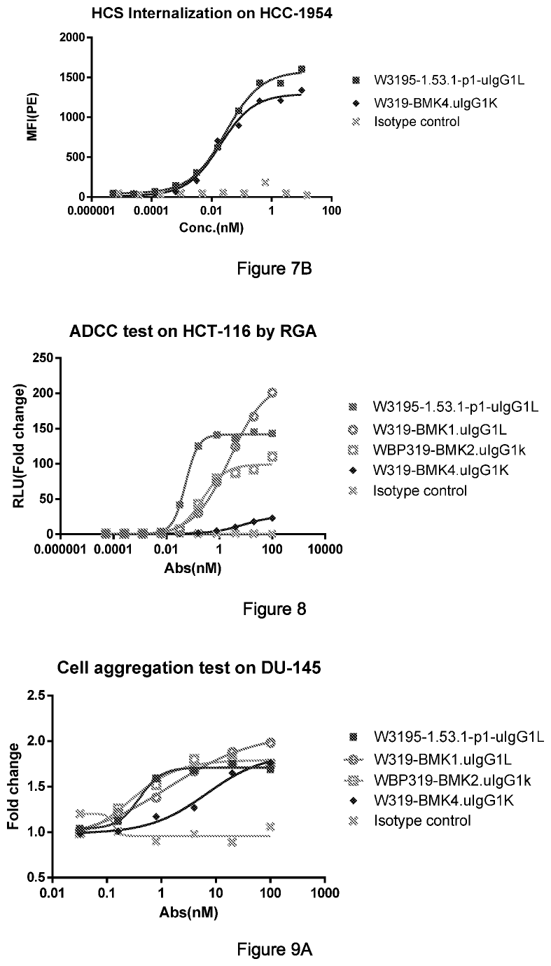

As indicated in Table 9 and FIG. 7A, W3195-1.53.1-p1-uIgG1L and W3195-1.53.1-p3-uIgG1L showed good internalization ability with an IC50 of 0.020 nM and 0.020 nM by Fab-ZAP CTG assay respectively, which was better than BMK4 (0.076 nM).

TABLE 9

HCC-1954

| mAbs | IC50 (nM) | Max Inh % |
|---|---|---|
| W3195-1.53.1-p1-uIgG1L | 0.020 | 81 |
| W3195-1.53.1-p3-uIgG1L | 0.020 | 78 |
| W319-BMK4.uIgG1K | 0.076 | 82 |
| Isotype control | >1 | 31 |
| Fab-ZAP only | >1 | 25 |

### 3.8 Antibody Mediated Internalization Assay (High Content Screening, HCS)

Operetta CLS (PerkinElmer) is a high content imaging and analysis system that can collect and analyze images of samples with high speed and sensitivity. One day before the assay day, Poly-D-Lysine (PDL) was diluted to 8 μg/mL in DPBS (Hyclone, #SH30028.03) and added 100 μL/well into 96-well clear bottom black plates (Greinier, #655090). The PDL-coated plates were then incubated at 37° C. for 1 hour before discarding the supernatant. HCC-1954 (ATCC, CRL-2338) cells were plated at 18000 cells per well into the PDL-coated plates in 100 μL RPMI1640 complete medium (Gibco, #22400-089) containing 10% FBS (Hyclone, #SH30084.03). On Day 1, supernatants in the plates were discarded and antibodies serially diluted in 1% BSA/1×PBS (Ca+/Mg+) were added at 100 μL/well and incubated at 4° C. for 2 hours. A non-related hIgG1 antibody was used as an isotype control. Cells were washed with 100 μL 1% BSA/1×PBS (Ca+/Mg+) by Multi-channel Pipettes (Eppendorf) and then resuspended in PE conjugated Goat anti-human IgG Fc (Jackson, #109-115-098) 1:150 diluted in 1% BSA/1×PBS (Ca+/Mg+) for 1 hour at 4° C. in the dark. Cells were then washed one time as described above and resuspended in 1% BSA/1×PBS (Ca+/Mg+) for 2 hours at 37° C. Supernatants were discarded and 100 μL/well quench buffer (0.1 M glycine, 0.15 M NaCl, adjust pH to 2.5) were added and incubated at 4° C. for 5 min. Cells were then washed one time as described above and resuspended in Hoechst 33342 (Invitrogen, #H3570) 1:5000 diluted in DPBS (Hyclone, #SH30028.03) for 15 min at room temperature. After washed with DPBS (Hyclone, #SH30028.03) one time as described above, cells were resuspended in 4% PFA and stored at 4° C. Images were collected and analyzed by operetta CLS (PerkinElmer). The amount of internalized anti-P-cadherin antibody was assessed by measuring the mean fluorescence (MFI) per cell, wells containing no antibody or secondary antibody only were used to establish background fluorescence. Internalization EC50 values were obtained by the four-parameter non-linear regression analysis using GraphPad Prism 6 software.

As indicated in Table 10 and FIG. 7B, W3195-1.53.1-p1-uIgG1L showed good internalization ability with an EC50 of 0.029 nM and a Top MFI of 1605 (higher than BMK4) using HCS assay.

TABLE 10

HCC-1954

| Abs | EC50 (nM) | Top MFI |
|---|---|---|
| W3195-1.53.1-p1-uIgG1L | 0.029 | 1605 |
| W319-BMK4.uIgG1K | 0.019 | 1339 |
| Isotype control | >10 | 184 |

### 3.9 Antibody-Dependent Cellular Cytotoxicity (ADCC) by Reporter Gene Assay (RGA)

The ADCC ability of anti-P-cadherin antibodies were measured using reporter gene assay (RGA). Jurkat-NFAT-CD16.A5 cell line was engineered using Jurkat (ATCC, #TIB-152) cells by stably expressing CD16-V158 protein and a luciferase reporter gene under the control of nuclear factor activated T-cells (NAFT) response element. In brief, on the day of ADCC, P-cadherin expressing cells HCT-116 (ATCC, #CCL-247, 8e4 cells/well) were plated in 96-well plate (Corning #3903) with Jurkat-NFAT-CD16.A5 cells (4e4 cells/well) and serial dilution of antibodies or hIgG isotype control in 100 μL RPMI1640 complete medium (Gibco, #22400-089) containing 10% fetal bovine serum (Hyclone, #SH30028.03). After incubating at 37° C. for nearly 4 h, 50 μL One-Glo luciferase assay system (Promega, #E6120) reagent was added to each well and incubated at room temperature for 10 minutes. Then plates were read by Envision (PerkinElmer) to measure luminescence signals. The ADCC activity of antibodies were expressed as fold change by comparing luminescence obtained with the control well without any antibody addition. The EC50 values were then calculated by four-parameter non-linear regression analysis using GraphPad Prism6 software.

As indicated in Table 11 and FIG. 8, W3195-1.53.1-p1-uIgG1L showed potent ADCC effect on hPro1 expressing HCT-116 cells using reporter gene assay (RGA), with an EC50 of 0.052 nM, which are much better than W319-BMK1, W319-BMK2 & W319-BMK4 (2.52, 0.23 & 8.05 nM).

TABLE 11

ADCC

| Abs | EC50 (nM) | Max Fold Change |
|---|---|---|
| W3195-1.53.1-p1-uIgG1L | 0.052 | 145 |
| W319-BMK1.uIgG1L | 2.52 | 201 |
| WBP319-BMK2.uIgG1K | 0.23 | 110 |
| W319-BMK4.uIgG1K | 8.05 | 23 |
| Isotype control | >100 | 1 |

### 3.10 Cell Aggregation Assay

The ability of antibodies to interfere with P-cadherin-dependent cell aggregation was measured using cell aggregation assay. In brief, DU-145 (ATCC, #HTB-81) cells were detached with Versene (Invitrogen, #15040066), resuspended in RPMI1640 (Gibco, #22400-089) complete medium with 10% fetal bovine serum (Hyclone, #SH30028.03) and seeded in 96-well plate (PerkinElmer, #6055330) at 1e4 cells/well with serial dilution of antibodies or hIgG isotype control in 100 μL medium. After incubating at 37° C. for 48 h, the plates were scanned using Operetta CLS (PerkinElmer) to measure areas of cell aggregation. The extent of cell aggregation disruption obtained with each antibody was quantified as fold change by comparing the area of aggregated cells obtained with the control well without any antibody addition. The EC50 values were calculated by four-parameter non-linear regression analysis using GraphPad Prism6 software.

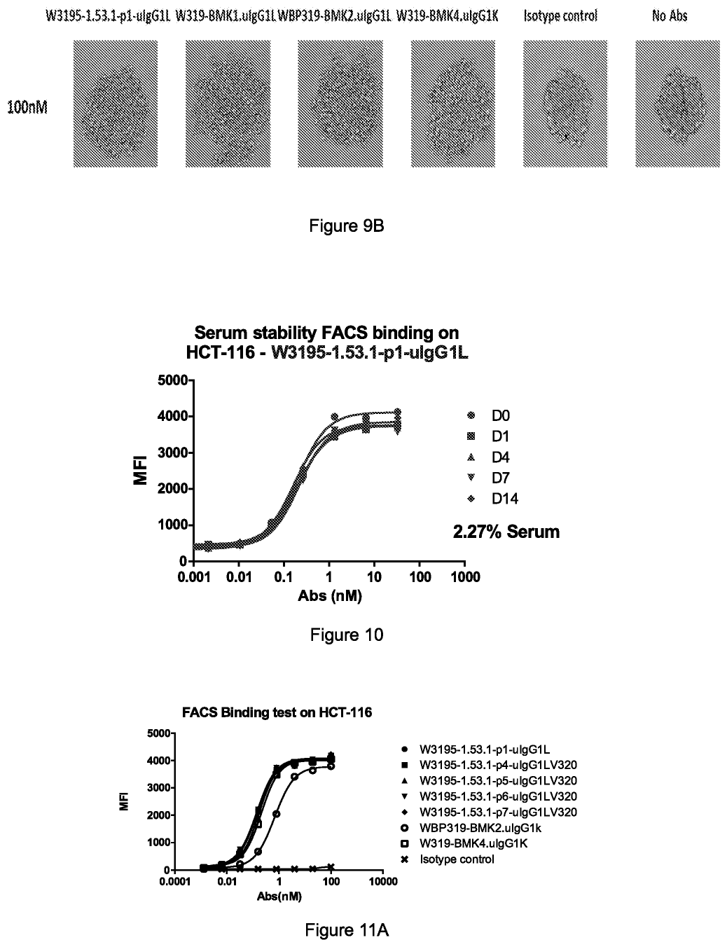

As indicated in Table 12 and FIG. 9A-B, W3195-1.53.1-p1-uIgG1L showed some effect to the aggregation of hPro1 expressing DU-145 cells, with an EC50 of 0.39 nM, which was better than W319-BMK1 & W319-BMK4 (1.37 & 6.79 nM), comparable with W319-BMK2 (0.26 nM).

TABLE 12

Fold change

| Abs | EC50 (nM) | Max Fold change |
|---|---|---|
| W3195-1.53.1-p1-uIgG1L | 0.39 | 1.75 |
| W319-BMK1.uIgG1L | 1.37 | 1.98 |
| WBP319-BMK2.uIgG1k | 0.26 | 1.80 |
| W319-BMK4.uIgG1K | 6.79 | 1.76 |
| Isotype control | >100 | 1.06 |

### 3.11 Non-Specific Binding (ELISA/FACS)

Both FACS and ELISA assays were used to test whether the antibodies bind to other human proteins.

### Protein-Based Non-Specific Binding by ELISA

ELISA plates (Nunc) were coated with human proteins at 2 μg/mL overnight at 4° C. After blocking and washing, 10 μg/ml antibody samples were added to the plates and incubated at room temperature for 2 h. The plates were then washed and subsequently incubated with goat anti human IgG-Fc HRP (Bethyl) for 1 h. After washing, TMB substrate was added and the interaction was stopped by 2M HCl. The absorbance at 450 nm was read using a microplate reader (Molecular Device).

### Cell-Based Non-Specific Binding by FACS

Human cell lines were seeded in 96-well plates (BD) at a density of 1×10⁵ cells/well. 10 μg/ml antibody samples were added to cells and incubated for 1 h at 4° C. After washing, the cells were resuspended and incubated with PE-conjugated goat anti-human IgG Fc antibody (Jackson) for 30 min. After washing and resuspending, fluorescence intensity was measured by flow cytometry (BD Canto II) and analyzed by FlowJo.

W3195-1.53.1-p1-uIgG1L showed no non-specific binding effect using proteins & cell based assay.

### 3.12 Thermo Stability by DSF

Tₘ of antibodies was investigated using QuantStudio™ 7 Flex Real-Time PCR system (Applied Biosystems). 19 μL of antibody solution was mixed with 1 μL of 62.5×SYPRO Orange solution (Invitrogen) and transferred to a 96 well plate (Biosystems). The plate was heated from 26° C. to 95° C. at a rate of 0.9° C./min, and the resulting fluorescence data was collected. The negative derivatives of the fluorescence changes with respect to different temperatures were calculated, and the maximal value was defined as melting temperature Tₘ. If a protein has multiple unfolding transitions, the first two Tₘ were reported, named as Tₘ1 and Tₘ2. Data collection and Tₘ calculation were conducted automatically by the operation software.

As indicated in Table 13, W3195-1.53.1-p1-uIgG1L showed Tm1 of 65.1° C. by DSF test.

TABLE 13

| | | | Concentration | Tm1 | Tm2 | |
|---|---|---|---|---|---|---|
| mAb | pl | Buffer | (mg/ml) | (° C.) | (° C.) | Comments |
| W3195-1.53.1-p1-uIgG1L | 8.1 | 20 mM His, 7% sucrose, pH 6.5 | 0.5 | 65.1 | — | Good |

### 3.13 Serum Stability

The antibody stability in human serum was tested by FACS. Briefly, human serum was freshly isolated by centrifuging the fresh blood twice at 4000 rpm for 10 minutes. Antibodies were mixed with freshly isolated human serum (serum content >95%) and incubated at 37° C. for 0, 1, 4, 7 and 14 days respectively, after which the samples were rapidly frozen in liquid nitrogen or dry ice/ethanol bath and stored at −80° C. until use. As a control, antibodies serum mix was frozen immediately without 37° C. incubation. For the FACS analysis, the samples from different time points were free-thawed simultaneously at 4° C. The thawed antibodies were serially diluted and added to 1×10⁵/well HCT-116 (ATCC, #CCL-247) cells and incubated for 1 hour at 4° C. The cells were washed twice with 1% BSA/1×PBS (Ca+/Mg+). Alexa647 conjugated Goat anti-human IgG Fc (Jackson, #109-605-098) 1:500 diluted in 1% BSA/1×PBS (Ca+/Mg+) were added to the cells and incubated at 4° C. for 30 minutes. Cells were washed twice in the same buffer and the mean fluorescence (MFI) of stained cells was measured using a FACS Canto II cytometer (BD Biosciences) and analyzed by FlowJo. Wells containing no antibody or secondary antibody only were used to establish background fluorescence. Four-parameter non-linear regression analysis was used to obtain EC50 values for cell binding using GraphPad Prism6 software.

As indicated in Table 14 and FIG. 10, W3195-1.53.1-p1-uIgG1L showed good stability as determined by comparable FACS binding after 14 days incubation in human serum (37° C.).

TABLE 14

| | Day 0 | | Day 1 | | Day 4 | | Day 7 | | Day 14 | |
|---|---|---|---|---|---|---|---|---|---|---|
| | EC50 | Max | EC50 | Max | EC50 | Max | EC50 | Max | EC50 | Max |
| mAb | (nM) | MFI | (nM) | MFI | (nM) | MFI | (nM) | MFI | (nM) | MFI |
| W3195-1.53.1-p1-uIgG1L | 0.20 | 4126 | 0.19 | 3789 | 0.16 | 3777 | 0.21 | 3785 | 0.20 | 3960 |

3.14 Function Assay after PTM Removal and Fc Modification

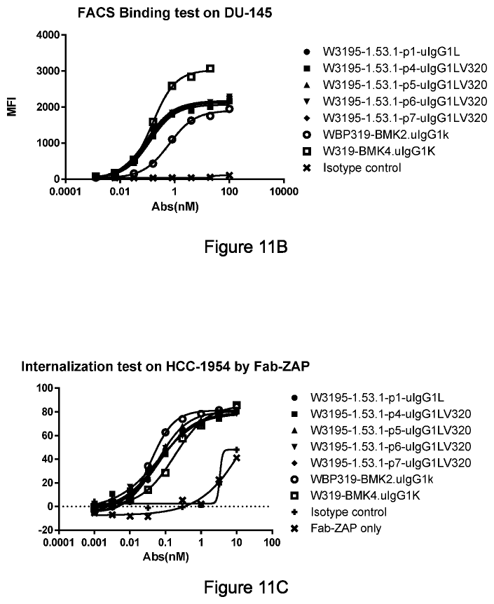

Binding of the antibodies to human P-cadherin expressed on cells was determined by flow cytometry analysis. After PTM removal and Fc modification, W3195-1.53.1-p4-uIgG1LV320, W3195-1.53.1-p5-uIgG1LV320, W3195-1.53.1-p6-uIgG1LV320 & W3195-1.53.1-p7-uIgG1LV320 showed good binding ability on hPro1 expressing HCT-116 cells, with EC50 0.14, 0.17, 0.14 & 0.14 nM respectively, which is comparable with W3195-1.53.1-p1-uIgG1L (0.15 nM) (FIG. 11A).

TABLE 15A

HCT-116

| Abs | EC50 (nM) | Max MFI |
|---|---|---|
| W3195-1.53.1-p1-uIgG1L | 0.15 | 4076 |
| W3195-1.53.1-p4-uIgG1LV320 | 0.14 | 4186 |
| W3195-1.53.1-p5-uIgG1LV320 | 0.17 | 4166 |
| W3195-1.53.1-p6-uIgG1LV320 | 0.14 | 4176 |
| W3195-1.53.1-p7-uIgG1LV320 | 0.14 | 4233 |
| WBP319-BMK2.uIgG1k | 0.69 | 3794 |
| W319-BMK4.uIgG1K | 0.21 | 4052 |
| Isotype control | >100 | 110 |

| | |
|---|---|
| Neg | 27 |
| Blank | 30 |

As indicated in the Table below and FIG. 11B, they also showed good binding ability on hPro1 expressing DU-145 cells, with EC50 0.12, 0.12, 0.10 & 0.09 nM respectively, which is comparable with W3195-1.53.1-p1-uIgG1L (0.10 nM).

TABLE 15B

DU-145

| Abs | EC50 (nM) | Max MFI |
|---|---|---|
| W3195-1.53.1-p1-uIgG1L | 0.10 | 2291 |
| W3195-1.53.1-p4-uIgG1LV320 | 0.12 | 2162 |
| W3195-1.53.1-p5-uIgG1LV320 | 0.12 | 2243 |
| W3195-1.53.1-p6-uIgG1LV320 | 0.10 | 2292 |
| W3195-1.53.1-p7-uIgG1LV320 | 0.09 | 2281 |
| WBP319-BMK2.uIgG1k | 0.62 | 1947 |
| W319-BMK4.uIgG1K | 0.15 | 3070 |
| Isotype control | >100 | 101 |

| | |
|---|---|
| Neg | 25 |
| Blank | 27 |

As shown in FIG. 11C, W3195-1.53.1-p4-uIgG1LV320, W3195-1.53.1-p5-uIgG1LV320, W3195-1.53.1-p6-uIgG1LV320 & W3195-1.53.1-p7-uIgG1LV320 showed good internalization ability on hPro1 expressing HCC-1954 cells, with EC50 0.063, 0.058, 0.063 & 0.059 nM respectively, which is comparable with W3195-1.53.1-p1-uIgG1L (0.069 nM).

TABLE 15C

HCC-1954

| Abs | IC50 (nM) | Max Inh % |
|---|---|---|
| W3195-1.53.1-p1-uIgG1L | 0.069 | 81 |
| W3195-1.53.1-p4-uIgG1LV320 | 0.063 | 80 |
| W3195-1.53.1-p5-uIgG1LV320 | 0.058 | 82 |
| W3195-1.53.1-p6-uIgG1LV320 | 0.063 | 79 |
| W3195-1.53.1-p7-uIgG1LV320 | 0.059 | 79 |
| WBP319-BMK2.uIgG1k | 0.039 | 84 |
| W319-BMK4.uIgG1K | 0.18 | 86 |
| Isotype control | >10 | 48 |
| Fab-ZAP only | >10 | 41 |

Those skilled in the art will further appreciate that the present disclosure may be embodied in other specific forms without departing from the spirit or central attributes thereof. In that the foregoing description of the present disclosure discloses only exemplary embodiments thereof, it is to be understood that other variations are contemplated as being within the scope of the present disclosure. Accordingly, the present disclosure is not limited to the particular embodiments that have been described in detail herein. Rather, reference should be made to the appended claims as indicative of the scope and content of the disclosure.

1. An isolated antibody or antigen-binding portion thereof that binds P-cadherin, wherein the isolated antibody or antigen-binding portion thereof comprises:
a HCDR1 comprising SEQ ID NO: 2;
a HCDR2 comprising SEQ ID NO: 4;
a HCDR3 comprising SEQ ID NO: 6, 8, 9, 10 or 11;
a LCDR1 comprising SEQ ID NO: 13;
a LCDR2 comprising SEQ ID NO: 15; and
a LCDR3 comprising SEQ ID NO: 17.

2. The isolated antibody or antigen-binding portion thereof of claim 1, wherein the isolated antibody or antigen-binding portion thereof comprises:
(A) a HCDR1 as set forth in SEQ ID NO: 2; a HCDR2 as set forth in SEQ ID NO: 4; and a HCDR3 as set forth in SEQ ID NO: 6; and
(B) a LCDR1 as set forth in SEQ ID NO: 13; a LCDR2 as set forth in SEQ ID NO: 15; and a LCDR3 as set forth in SEQ ID NO: 17.

3. (canceled)

4. The isolated antibody or antigen-binding portion thereof of claim 1, wherein the isolated antibody or antigen-binding portion thereof comprises:
(A) a heavy chain variable region (VH):
(i) comprising the amino acid sequence as set forth in SEQ ID NO: 21;
(ii) comprising an amino acid sequence at least 85%, 90%, or 95% identical to the amino acid sequence as set forth in SEQ ID NO: 21 yet retaining the specific binding affinity to P-cadherin; or
(iii) comprising an amino acid sequence with addition, deletion and/or substitution of one or more amino acids in the framework region compared with the amino acid sequence as set forth in SEQ ID NO: 21; and/or
(B) a light chain variable region (VL):
(i) comprising the amino acid sequence as set forth in SEQ ID NO: 27;
(ii) comprising an amino acid sequence at least 85%, at least 90%, or at least 95% identical to the amino acid sequence as set forth in SEQ ID NO: 27 yet retaining the specific binding affinity to P-cadherin; or
(iii) comprising an amino acid sequence with addition, deletion and/or substitution of one or more amino acids in the framework region compared with the amino acid sequence as set forth in SEQ ID NO: 27.

5. The isolated antibody or antigen-binding portion thereof of claim 4, comprising one or more substitutions of amino acids in any of FRW1, FRW2, FRW3, and/or FRW4 of the VH or VL region.

6. The isolated antibody or antigen-binding portion thereof of claim 1, comprising a heavy chain variable region (VH) and a light chain variable region (VL), wherein:
the VH comprises one or more heavy chain FRWs (HFRWs) selected from the group consisting of:
(i) a HFRW1 comprising SEQ ID NO: 1;
(ii) a HFRW2 comprising SEQ ID NO: 3;
(iii) a HFRW3 comprising SEQ ID NO: 5; and
(iv) a HFRW4 comprising SEQ ID NO: 7; and
the VL comprises one or more light chain FRWs (LFRWs) selected from the group consisting of:
(i) a LFRW1 comprising SEQ ID NO: 12, 19 or 20;
(ii) a LFRW2 comprising SEQ ID NO: 14;
(iii) a LFRW3 comprising SEQ ID NO: 16; and
(iv) a LFRW4 comprising SEQ ID NO: 18.

7. The isolated antibody or antigen-binding portion thereof of claim 1, wherein the isolated antibody or antigen-binding portion thereof comprises a heavy chain variable region comprising the amino acid sequence as set forth in SEQ ID NO: 21, 22, 23, 24 or 25; and a light chain variable region comprising the amino acid sequence as set forth in SEQ ID NO: 26, 27 or 28.

8. The isolated antibody or antigen-binding portion thereof of claim 1, wherein the isolated antibody or antigen-binding portion thereof further comprises a human IgG constant domain.

9. The isolated antibody or antigen-binding portion thereof of claim 8, wherein the human IgG constant domain is a human IgG1, IgG2, IgG3 or IgG4 constant domain, preferably a human IgG1 constant domain or a variant thereof, such as a variant comprising L234A and L235A substitutions, according to EU numbering.

10. The isolated antibody or antigen-binding portion thereof of claim 1, which has one or more of the following properties:
(a) bind to cell surface human P-cadherin or cynomolgus monkey P-cadherin with an EC50 in nM grade, as measured by FACS;
(b) bind to cell surface human P-cadherin with a KD no more than 0.1 nM, as measured by FACS affinity test;
(c) have no cross-reactivity to human E-cadherin or N-cadherin;
(d) have good internalization ability comparable with benchmark antibodies;
(e) have significantly better ADCC effect than benchmark antibodies;
(f) inhibit the aggregation of human P-cadherin expressing cells with an EC50 in nM grade;
(g) show no non-specific binding effect; and
(h) being stable in serum for at least 14 days.

11. (canceled)

12. The isolated antibody or antigen-binding portion thereof of claim 1, wherein the antibody is a chimeric antibody, a humanized antibody or a fully human antibody, preferably is a fully human monoclonal antibody.

13. The isolated antibody or antigen-binding portion thereof of claim 1, wherein:
(a) the heavy chain of the antibody comprises a heavy chain variable region as set forth in SEQ ID NO: 21, 22, 23, 24 or 25, and a heavy chain constant region as set forth in SEQ ID NO: 29 or 31; and
(b) the light chain of the antibody comprises a light chain variable region as set forth in SEQ ID NO: 26, 27 or 28, and a light chain constant region as set forth in SEQ ID NO: 30.

14. An isolated nucleic acid molecule, comprising a nucleic acid sequence encoding the heavy chain variable region and/or the light chain variable region of the isolated antibody as defined in claim 1.

15. A vector comprising the nucleic acid molecule of claim 14.

16. A host cell comprising the vector of claim 15.

17. A pharmaceutical composition comprising at least one antibody or antigen-binding portion thereof as defined in claim 1 and a pharmaceutically acceptable carrier.

18. A method for producing the antibody or antigen-binding portion thereof as defined in claim 1 comprising the steps of:
culturing a host cell comprising a vector(s) encoding the antibody or antigen-binding portion thereof under suitable conditions; and
isolating the antibody or antigen-binding portion thereof from the culture supernatant.

19. (canceled)

20. A method for treating or preventing P-cadherin positive cancer in a subject, comprising administering an effective amount of the antibody or antigen-binding portion thereof as defined in claim 1 to the subject.

21. The method of claim 20, wherein the cancer is selected from cholangio carcinomas, esophageal cancer, oral cancers, thyroid tumor, head and neck cancers, breast cancer, lung cancers, malignant mesothelioma, colorectal cancer, ovarian cancer, cervical cancer, melanoma, skin cancers, bladder cancer, liver cancer, prostate cancer, stomach cancer, kidney cancers, pancreatic cancer, endometrial cancer, urothelial cancer, sarcomas, osteosarcoma, and bone cancers.

22. The method of claim 21, wherein the cancer is breast cancer, NSCLC, prostate cancer or colorectal cancer.

23-24. (canceled)

25. A kit for treating or diagnosing cancer, comprising a container comprising the antibody or antigen-binding portion thereof as defined in claim 1.


SEQUENCE LISTING (31 sequences)

SEQ ID NO: 1 - protein, 25 aa, Artificial sequence
  VH FRW1
QVQLQESGPGLVKPSETLSLTCTVS

SEQ ID NO: 2 - protein, 12 aa, Artificial sequence
  HCDR1
GGSVISDNYYWT

SEQ ID NO: 3 - protein, 14 aa, Artificial sequence
  VH FRW2
WVRQPPGKGLEFIG

SEQ ID NO: 4 - protein, 16 aa, Artificial sequence
  HCDR2
YIYYRGSTNYNPSLKN

SEQ ID NO: 5 - protein, 32 aa, Artificial sequence
  VH FRW3
RVIISIDTSRNQFSLDLNSVTAADTAVYYCAR

SEQ ID NO: 6 - protein, 12 aa, Artificial sequence
  HCDR3
DRRTGNSLPFDN

SEQ ID NO: 7 - protein, 11 aa, Artificial sequence
  VH FRW4
WGQGTLVTVSS

SEQ ID NO: 8 - protein, 12 aa, Artificial sequence
  HCDR3
DRRTGNALPFDN

SEQ ID NO: 9 - protein, 12 aa, Artificial sequence
  HCDR3
DRRTGQSLPFDN

SEQ ID NO: 10 - protein, 12 aa, Artificial sequence
  HCDR3
DRRTGTSLPFDN

SEQ ID NO: 11 - protein, 12 aa, Artificial sequence
  HCDR3
DRRTGSSLPFDN

SEQ ID NO: 12 - protein, 22 aa, Artificial sequence
  VL FRW1
SYELTQPPSVSVSPGQTANITC

SEQ ID NO: 13 - protein, 11 aa, Artificial sequence
  LCDR1
SGDKLGDKFAS

SEQ ID NO: 14 - protein, 15 aa, Artificial sequence
  VL FRW2
WYQQKSGQSPVVVIY

SEQ ID NO: 15 - protein, 7 aa, Artificial sequence
  LCDR2
QDNKRPS

SEQ ID NO: 16 - protein, 32 aa, Artificial sequence
  VL FRW3
GFPERFSGSNSGNTATLTISGTQAMDEADYYC

SEQ ID NO: 17 - protein, 9 aa, Artificial sequence
  LCDR3
QAWDSSIVV

SEQ ID NO: 18 - protein, 10 aa, Artificial sequence
  VL FRW4
FGGGTKLTVL

SEQ ID NO: 19 - protein, 22 aa, Artificial sequence
  VL FRW1
SYELTQPPSVSVSPGQTAQITC

SEQ ID NO: 20 - protein, 22 aa, Artificial sequence
  VL FRW1
SYELTQPPSVSVSPGQTANIAC

SEQ ID NO: 21 - protein, 122 aa, Artificial sequence
  VH
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGNSLPFDNWGQGTLVTVSS

SEQ ID NO: 22 - protein, 122 aa, Artificial sequence
  VH
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGNALPFDNWGQGTLVTVSS

SEQ ID NO: 23 - protein, 122 aa, Artificial sequence
  VH
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGQSLPFDNWGQGTLVTVSS

SEQ ID NO: 24 - protein, 122 aa, Artificial sequence
  VH
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGTSLPFDNWGQGTLVTVSS

SEQ ID NO: 25 - protein, 122 aa, Artificial sequence
  VH
QVQLQESGPGLVKPSETLSLTCTVSGGSVISDNYYWTWVRQPPGKGLEFIGYIYYRGSTNYNPSLKNRVIISIDTSRNQFSLDLNSVTAADTAVYYCARDRRTGSSLPFDNWGQGTLVTVSS

SEQ ID NO: 26 - protein, 106 aa, Artificial sequence
  VL
SYELTQPPSVSVSPGQTANITCSGDKLGDKFASWYQQKSGQSPVVVIYQDNKRPSGFPERFSGSNSGNTATLTISGTQAMDEADYYCQAWDSSIVVFGGGTKLTVL

SEQ ID NO: 27 - protein, 106 aa, Artificial sequence
  VL
SYELTQPPSVSVSPGQTAQITCSGDKLGDKFASWYQQKSGQSPVVVIYQDNKRPSGFPERFSGSNSGNTATLTISGTQAMDEADYYCQAWDSSIVVFGGGTKLTVL

SEQ ID NO: 28 - protein, 106 aa, Artificial sequence
  VL
SYELTQPPSVSVSPGQTANIACSGDKLGDKFASWYQQKSGQSPVVVIYQDNKRPSGFPERFSGSNSGNTATLTISGTQAMDEADYYCQAWDSSIVVFGGGTKLTVL

SEQ ID NO: 29 - protein, 330 aa, Artificial sequence
  HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 106 aa, Artificial sequence
  LC constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 330 aa, Artificial sequence
  HC constant region, V320
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK